SEQUENCE LISTING (60 sequences)

SEQ ID NO: 1 - protein, 394 aa, Homo sapiens
  mAAT (1)..(394): modified human AAT without signal sequence with original 256 Cys mutated to Ser (232)
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 394 aa, Homo sapiens
  Human AAT (1)..(394): Original human AAT without signal sequence MPSSVSWGILLLAGLCCLVPVSLA
  Human AAT_WithoutSignalSeq (1)..(394): Original human AAT without signal sequence MPSSVSWGILLLAGLCCLVPVSLA
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 418 aa, Homo sapiens
  Human_AAT_SignqlSeq (1)..(24): Human AAT signal sequence
  Human_AAT (1)..(418): Human AAT full sequence sp|P01009|A1AT_HUMAN Alpha-1-antitrypsin OS=Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - protein, 153 aa, Homo sapiens
  IL2_Signal (1)..(20): Original Human IL-2 with signal sequence
  IL2_HumanFull (1)..(153): Original Human IL-2 with signal sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 5 - protein, 534 aa, Homo sapiens
  IL2 (1)..(134): Human IL-2 with C126 to S126 with added first M
  mIL2_mAAT_ShortLinker (1)..(534): Human IL-2 with C126 to S126 with added first M_Short Linker_mAAT
  IL2 (1)..(134): Human IL-2 with C126 to S126 with added first M
  Linker (135)..(140): Linker between IL-2 and mAAT Short Linker GSTSGS
  mAAT (141)..(534): Human mAAT with Cys372 mutation to Ser 372
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGSTSGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 6 - protein, 538 aa, Homo sapiens
  IL2 (1)..(134): Human IL2 with 126 Cys to Ser mutation
  mIL2_mAAT_LongLinker (1)..(538): Human IL2 with 126 Cys to Ser mutation
  IL2 (1)..(134): Human IL2 with 126 Cys to Ser mutation
  Linker (135)..(144): Long liker GGGGSGGGGS
  mAAT (145)..(538): Human AAT without signal sequence and with Cys256 (376) to Ser mutation
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 7 - DNA, 1605 bp, Homo sapiens
  mIL2_mAAT_Short (1)..(1605): Short linker with X=Ser, Z=Ser mutations
atggctcccacgtcgagtagtactaaaaaaactcagcttcagttagaacatctgttgttggatttgcagatgatcttgaacggtattaacaactataagaatccgaagttgacgcgcatgcttacgttcaagttctacatgcccaagaaagctacggagctgaaacatttacagtgtttggaagaagaactgaagccgttggaggaggtattaaatttggcacaatctaagaattttcatttacgcccacgtgatctgattagtaatatcaacgtcatcgtattggagctgaagggcagtgagacgacattcatgtgtgagtatgccgacgaaacagctacgattgtagaatttcttaatcgttggattaccttctctcagtctatcatttcaaccttaactggctctacgtccgggtcggaagatcctcaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 8 - DNA, 1617 bp, Homo sapiens
  mIL2_mAAT_LongLinker (1)..(1617): DNA for mIL2 mAAT with mutations X=Ser Z=Ser, Long linker
atggctcccacgtcgagtagtactaaaaaaactcagcttcagttagaacatctgttgttggatttgcagatgatcttgaacggtattaacaactataagaatccgaagttgacgcgcatgcttacgttcaagttctacatgcccaagaaagctacggagctgaaacatttacagtgtttggaagaagaactgaagccgttggaggaggtattaaatttggcacaatctaagaattttcatttacgcccacgtgatctgattagtaatatcaacgtcatcgtattggagctgaagggcagtgagacgacattcatgtgtgagtatgccgacgaaacagctacgattgtagaatttcttaatcgttggattaccttctctcagtctatcatttcaaccttaactggcggtggtggctctggcggtggtggctccgaagatcctcaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 9 - protein, 14 aa, Homo sapiens
  IL2_Cyc125 (6)..(6): Cys original. X=C
RWITFCQSIISTLT

SEQ ID NO: 10 - protein, 14 aa, Homo sapiens
  mIL2_Ser125 (6)..(6): mIL2 with Cys125 mutation to Ser125. X=S
RWITFSQSIISTLT

SEQ ID NO: 11 - protein, 14 aa, Homo sapiens
  mIL2_Ala125 (6)..(6): human IL-2 with Cys125 mutation to Ala125. X=A
RWITFAQSIISTLT

SEQ ID NO: 12 - protein, 13 aa, Homo sapiens
  AAT_C256 (5)..(5): Human AAT with original Cys256. Z=C
NIQHCKKLSSWVL

SEQ ID NO: 13 - protein, 13 aa, Homo sapiens
  mAAT_Ser256 (5)..(5): Modified human AAT with Cys256 to Ser256 mutation. Z=S
NIQHSKKLSSWVL

SEQ ID NO: 14 - protein, 13 aa, Homo sapiens
  mAAT_Ala256 (5)..(5): Modified human AAT with Cys256 to Ala256 mutation. Z=A
NIQHAKKLSSWVL

SEQ ID NO: 15 - protein, 6 aa, Artificial Sequence
  Linker
GSTSGS

SEQ ID NO: 16 - protein, 10 aa, Artificial Sequence
  Linker
GGGGSGGGGS

SEQ ID NO: 17 - DNA, 31 bp, Artificial Sequence
  Primer
gttggattaccttctgtcagtctatcatttc

SEQ ID NO: 18 - DNA, 31 bp, Artificial Sequence
  Primer
gaaatgatagactgacagaaggtaatccaac

SEQ ID NO: 19 - DNA, 31 bp, Artificial Sequence
  Primer
tcaacatccaacactgcaagaaactgtcgtc

SEQ ID NO: 20 - DNA, 31 bp, Artificial Sequence
  Primer
gacgacagtttcttgcagtgttggatgttga

SEQ ID NO: 21 - DNA, 31 bp, Artificial Sequence
  Primer
cgttggattaccttcgctcagtctatcattt

SEQ ID NO: 22 - DNA, 31 bp, Artificial Sequence
  Primer
aaatgatagactgagcgaaggtaatccaacg

SEQ ID NO: 23 - DNA, 32 bp, Artificial Sequence
  Primer
ttcaacatccaacacgccaagaaactgtcgtc

SEQ ID NO: 24 - DNA, 32 bp, Artificial Sequence
  Primer
gacgacagtttcttggcgtgttggatgttgaa

SEQ ID NO: 25 - DNA, 1560 bp, Homo sapiens
  IL15(73D)-linker-AAT(Z=Ser) cDNA (1)..(1560)
atgaactgggtgaatgtaatatctgatttaaagaagatagaagaccttattcagagtatgcacatagatgctacgctttatacggagtccgatgtgcaccctagttgcaaggtgacggcgatgaagtgctttttacttgaattgcaagttatttcccttgaatcgggggacgccagtatacacgacacagtggaaaatttgattatcctggctaacgatagcctgtcgagcaacggaaatgtgacagaaagtggatgtaaggagtgcgaggagttagaggaaaagaacattaaagagttccttcaatcattcgtgcatatcgtccagatgttcattaacacatcaggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 26 - protein, 519 aa, Homo sapiens
  IL15(73D)-linker-AAT(Z=Ser) protein (1)..(519)
MNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 27 - DNA, 1560 bp, Homo sapiens
  IL15(73N)-linker-AAT(Z=Ser) cDNA (1)..(1560)
atgaactgggtgaatgtaatatctgatttaaagaagatagaagaccttattcagagtatgcacatagatgctacgctttatacggagtccgatgtgcaccctagttgcaaggtgacggcgatgaagtgctttttacttgaattgcaagttatttcccttgaatcgggggacgccagtatacacgacacagtggaaaatttgattatcctggctaacaatagcctgtcgagcaacggaaatgtgacagaaagtggatgtaaggagtgcgaggagttagaggaaaagaacattaaagagttccttcaatcattcgtgcatatcgtccagatgttcattaacacatcaggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 28 - protein, 519 aa, Homo sapiens
  IL15(73N)-linker-AAT(Z=Ser) protein (1)..(519)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 29 - DNA, 1560 bp, Homo sapiens
  IL15(73N)-linker-AAT(Z=Cys) cDNA (1)..(1560)
atgaactgggtgaatgtaatatctgatttaaagaagatagaagaccttattcagagtatgcacatagatgctacgctttatacggagtccgatgtgcaccctagttgcaaggtgacggcgatgaagtgctttttacttgaattgcaagttatttcccttgaatcgggggacgccagtatacacgacacagtggaaaatttgattatcctggctaacaatagcctgtcgagcaacggaaatgtgacagaaagtggatgtaaggagtgcgaggagttagaggaaaagaacattaaagagttccttcaatcattcgtgcatatcgtccagatgttcattaacacatcaggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 30 - protein, 519 aa, Homo sapiens
  IL15(73N)-linker-AAT(Z=Cys) protein (1)..(519)
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 31 - DNA, 1740 bp, Homo sapiens
  G-CSF-Linker-AAT(Z=Ser) cDNA (1)..(1740)
atgacacccttgggcccagcaagctctctgcctcaatcttttttacttaaaagtcttgaacaggtgcggaagattcaaggagatggggcagcacttcaagaaaaattgtgtgctacgtacaaactgtgtcatccagaagaattagtgttactgggacattctcttgggataccgtgggcgccgctttctagctgtccaagtcaagcgttacagcttgcgggatgcctgtcgcagttgcactcaggtctgttcttgtaccaaggacttcttcaggcattggaagggatctcccctgaactggggcctactttggacactttgcagttagacgtagcggattttgcaacgactatctggcagcagatggaagagctgggcatggcaccagcgttacaaccaacgcaaggtgcgatgcccgccttcgcatcagcattccaacgtagagccggtggggttctggttgcttcgcaccttcaaagttttcttgaggtctcttatcgtgttctgagacatttagctcaaccaggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 32 - protein, 579 aa, Homo sapiens
  G-CSF-Linker-AAT(Z=Ser) protein (1)..(579)
MTPLGPASSLPQSFLLKSLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 33 - DNA, 1740 bp, Homo sapiens
  G-CSF-Linker-AAT(Z=Cys) cDNA (1)..(1740)
atgacacccttgggcccagcaagctctctgcctcaatcttttttacttaaaagtcttgaacaggtgcggaagattcaaggagatggggcagcacttcaagaaaaattgtgtgctacgtacaaactgtgtcatccagaagaattagtgttactgggacattctcttgggataccgtgggcgccgctttctagctgtccaagtcaagcgttacagcttgcgggatgcctgtcgcagttgcactcaggtctgttcttgtaccaaggacttcttcaggcattggaagggatctcccctgaactggggcctactttggacactttgcagttagacgtagcggattttgcaacgactatctggcagcagatggaagagctgggcatggcaccagcgttacaaccaacgcaaggtgcgatgcccgccttcgcatcagcattccaacgtagagccggtggggttctggttgcttcgcaccttcaaagttttcttgaggtctcttatcgtgttctgagacatttagctcaaccaggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 34 - protein, 579 aa, Homo sapiens
  G-CSF-Linker-AAT(Z=Cys) protein (1)..(579)
MTPLGPASSLPQSFLLKSLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 35 - DNA, 1599 bp, Homo sapiens
  GM-CSF-Linker-AAT(Z=Ser) cDNA (1)..(1599)
atggctccggcacggtcgccaagcccgagtacccaaccctgggagcatgttaatgcgattcaagaggcacggcgtcttcttaatttaagccgtgatacagcggcagagatgaatgaaacagtcgaggttatatcggaaatgtttgatcttcaggaacccacctgcctgcaaactagattggaattatacaaacaaggacttcgtgggagcctgacgaagctgaaggggcctttaactatgatggcatcacactacaagcaacattgtccgcccactcctgagacctcttgcgctacccagatcatcactttcgagtctttcaaggagaaccttaaagacttccttctggtaattcctttcgattgttgggagccggtgcaagagggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 36 - protein, 532 aa, Homo sapiens
  GM-CSF-linker-AAT(Z=Ser) protein (1)..(532)
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 37 - DNA, 1599 bp, Homo sapiens
  GM-CSF-Linker-AAT(Z=Cys)cDNA (1)..(1599)
atggctccggcacggtcgccaagcccgagtacccaaccctgggagcatgttaatgcgattcaagaggcacggcgtcttcttaatttaagccgtgatacagcggcagagatgaatgaaacagtcgaggttatatcggaaatgtttgatcttcaggaacccacctgcctgcaaactagattggaattatacaaacaaggacttcgtgggagcctgacgaagctgaaggggcctttaactatgatggcatcacactacaagcaacattgtccgcccactcctgagacctcttgcgctacccagatcatcactttcgagtctttcaaggagaaccttaaagacttccttctggtaattcctttcgattgttgggagccggtgcaagagggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 38 - protein, 532 aa, Homo sapiens
  GM-CSF-linker-AAT(Z=Cys) protein (1)..(532)
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 39 - DNA, 1713 bp, Homo sapiens
  IFN-a2-linker-AAT(Z=Ser) cDNA (1)..(1713)
atgtgcgatcttcctcagactcatagccttgggtcccggagaacgctgatgttgctggcccaaatgcgtcgcataagtcttttttcctgtcttaaagaccggcacgactttgggttcccccaggaggagtttgggaaccaatttcaaaaggctgagactattccggtcttacatgagatgatccaacagatattcaatttgttctccaccaaggactcatctgctgcttgggatgaaacgctgttagataagttttacacggagctttatcagcaactgaacgatttggaagcgtgtgtgatacaaggagtcggggttactgaaaccccgttaatgaaggaggacagcattcttgctgttcgcaaatattttcaacgtataactttgtatttgaaggagaagaaatattccccatgtgcatgggaggtcgtcagagcagaaattatgcgcagtttctcgttaagcactaatctgcaagagtctttgcgctcgaaagagggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 40 - protein, 570 aa, Homo sapiens
  IFN-a2-linker-AAT(Z=Ser) protein (1)..(570)
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 41 - DNA, 1713 bp, Homo sapiens
  IFN-a2-linker-AAT(Z=Cys) cDNA (1)..(1713)
atgtgcgatcttcctcagactcatagccttgggtcccggagaacgctgatgttgctggcccaaatgcgtcgcataagtcttttttcctgtcttaaagaccggcacgactttgggttcccccaggaggagtttgggaaccaatttcaaaaggctgagactattccggtcttacatgagatgatccaacagatattcaatttgttctccaccaaggactcatctgctgcttgggatgaaacgctgttagataagttttacacggagctttatcagcaactgaacgatttggaagcgtgtgtgatacaaggagtcggggttactgaaaccccgttaatgaaggaggacagcattcttgctgttcgcaaatattttcaacgtataactttgtatttgaaggagaagaaatattccccatgtgcatgggaggtcgtcagagcagaaattatgcgcagtttctcgttaagcactaatctgcaagagtctttgcgctcgaaagagggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 42 - protein, 570 aa, Homo sapiens
  IFN-a2-linker-AAT(Z=Cys) protein (1)..(570)
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 43 - DNA, 1713 bp, Homo sapiens
  IFN-b1-linker-AAT(Z=Ser) cDNA (1)..(1713)
atgagttataacttattgggtttcttgcaacggagttccaactttcagtcgcaaaaactgttgtggcagcttaatgggagattggaatattgcttgaaggaccgcatgaatttcgacatacctgaagaaattaaacaacttcagcagtttcagaaggaggatgcagctttaactatctacgaaatgttacaaaacatattcgcaatttttcgtcaggactcaagtagtacgggttggaacgaaacaatcgtagagaatttgttagccaacgtatatcatcagataaatcacttaaaaacagtacttgaagaaaaactggagaaggaggacttcacacgcgggaaacttatgagttcgcttcacttaaagcggtattacggacgcatcttacactacttgaaggctaaggaatattcccactgtgcctggacgatcgtgcgtgtcgagattcttcgtaatttctacttcataaaccgccttacaggatatttacggaatggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 44 - protein, 570 aa, Homo sapiens
  IFN-b1-linker-AAT(Z=Ser) protein (1)..(570)
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 45 - DNA, 1713 bp, Homo sapiens
  IFN-b1-linker-AAT(Z=Cys) cDNA (1)..(1713)
atgagttataacttattgggtttcttgcaacggagttccaactttcagtcgcaaaaactgttgtggcagcttaatgggagattggaatattgcttgaaggaccgcatgaatttcgacatacctgaagaaattaaacaacttcagcagtttcagaaggaggatgcagctttaactatctacgaaatgttacaaaacatattcgcaatttttcgtcaggactcaagtagtacgggttggaacgaaacaatcgtagagaatttgttagccaacgtatatcatcagataaatcacttaaaaacagtacttgaagaaaaactggagaaggaggacttcacacgcgggaaacttatgagttcgcttcacttaaagcggtattacggacgcatcttacactacttgaaggctaaggaatattcccactgtgcctggacgatcgtgcgtgtcgagattcttcgtaatttctacttcataaaccgccttacaggatatttacggaatggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 46 - protein, 570 aa, Homo sapiens
  IFN-b1-linker-AAT(Z=Cys) protein (1)..(570)
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 47 - DNA, 1311 bp, Homo sapiens
  mGLP1-linker-AAT(Z=Ser) cDNA (1)..(1311)
atgcatggggaagggacatttacaagtgacgtttcaagctacttggagggacaagccgcaaaggaattcatcgcctggctggtcaaggggagaggcggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 48 - protein, 436 aa, Homo sapiens
  mGLP1-linker-AAT(Z=Ser) protein (1)..(436)
MHGEGTFTSDVSSYLEGQAAKEFIAWLVKGRGGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 49 - DNA, 1311 bp, Homo sapiens
  mGLP1-linker-AAT(Z=Cys) cDNA (1)..(1311)
atgcatggggaagggacatttacaagtgacgtttcaagctacttggagggacaagccgcaaaggaattcatcgcctggctggtcaaggggagaggcggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 50 - protein, 436 aa, Homo sapiens
  mGLP1-linker-AAT(Z=Cys) protein (1)..(436)
MHGEGTFTSDVSSYLEGQAAKEFIAWLVKGRGGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 51 - DNA, 1764 bp, Homo sapiens
  AAT(Z=Ser)-linker-FGF21 cDNA (1)..(1764)
atggcggaagatcctcaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaaggcggtggtggatccggcggtggtggctcccatccgatccccgatagctcgccgctgctgcaatttggcgggcaagtgcgccaacgctacctgtacacggatgacgcacagcaaacagaggctcatttagaaatccgtgaggatggtactgtgggaggggcagccgatcagagtccggagtcattgttgcaactgaaagcattgaaacctggggtcattcagattttgggggtgaaaacaagccgctttttgtgccaacgccccgacggcgcgttgtacggtagcctgcacttcgaccctgaagcgtgttctttccgtgaattactgcttgaggatggttataatgtttatcaatcagaggcgcacgggctgccgctgcaccttcctggtaataaatcgccccaccgtgatccagctccacgcggaccagctcgtttcttaccacttccagggttgcctcctgcgcttcctgagccaccaggtatcctggctccccaaccgccagatgtcggctcttccgaccctttgagcatggtcggtccatcgcagggacgctcaccctcctacgcgagttaa

SEQ ID NO: 52 - protein, 587 aa, Homo sapiens
  AAT(Z=Ser)-linker-FGF21 protein (1)..(587)
MAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 53 - DNA, 1764 bp, Homo sapiens
  AAT(Z=Cys)-linker-FGF21 cDNA (1)..(1764)
atggcggaagatcctcaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaaggcggtggtggatccggcggtggtggctcccatccgatccccgatagctcgccgctgctgcaatttggcgggcaagtgcgccaacgctacctgtacacggatgacgcacagcaaacagaggctcatttagaaatccgtgaggatggtactgtgggaggggcagccgatcagagtccggagtcattgttgcaactgaaagcattgaaacctggggtcattcagattttgggggtgaaaacaagccgctttttgtgccaacgccccgacggcgcgttgtacggtagcctgcacttcgaccctgaagcgtgttctttccgtgaattactgcttgaggatggttataatgtttatcaatcagaggcgcacgggctgccgctgcaccttcctggtaataaatcgccccaccgtgatccagctccacgcggaccagctcgtttcttaccacttccagggttgcctcctgcgcttcctgagccaccaggtatcctggctccccaaccgccagatgtcggctcttccgaccctttgagcatggtcggtccatcgcagggacgctcaccctcctacgcgagttaa

SEQ ID NO: 54 - protein, 587 aa, Homo sapiens
  AAT(Z=Cys)-linker-FGF21 protein (1)..(587)
MAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQKGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 55 - DNA, 1602 bp, Homo sapiens
  sdAb-linker-AAT (Z=Ser)cDNA (1)..(1602)
atggaagtacagttagtggaatccggaggaggtctggtgcagcctgggggttccctgcgtttgtcatgcgcggcttcgggacgtactttctcgtacaacccaatgggctggtttcgtcaggcgcccggaaaaggccgggaattggttgccgctatcagtcgcactgggggatctacttattacccggatagcgtcgaaggacggtttaccatctcgcgggacaacgcaaagcggatggtttacttgcaaatgaactccctgcgtgcagaagacaccgcggtctactattgcgcggcggcaggtgtcagagctgaggacgggcgggtacggacgttgccctctgagtacaccttctggggtcagggaactcaagtgacagtgtcgagcggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactccaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 56 - protein, 533 aa, Homo sapiens
  sdAb-linker-AAT(Z=Ser) protein (1)..(533)
MEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHSKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 57 - DNA, 1602 bp, Homo sapiens
  sdAb-linker-AAT (Z=Cys)cDNA (1)..(1602)
atggaagtacagttagtggaatccggaggaggtctggtgcagcctgggggttccctgcgtttgtcatgcgcggcttcgggacgtactttctcgtacaacccaatgggctggtttcgtcaggcgcccggaaaaggccgggaattggttgccgctatcagtcgcactgggggatctacttattacccggatagcgtcgaaggacggtttaccatctcgcgggacaacgcaaagcggatggtttacttgcaaatgaactccctgcgtgcagaagacaccgcggtctactattgcgcggcggcaggtgtcagagctgaggacgggcgggtacggacgttgccctctgagtacaccttctggggtcagggaactcaagtgacagtgtcgagcggtggtggtggctctggcggtggtggctccgaagatccacaaggtgatgctgcgcaaaagaccgacacatcacaccacgatcaagatcatccaacatttaacaaaattacgcctaacttggccgagtttgcattcagtttgtatcgtcagcttgcgcatcaatccaattcaacaaatattttctttagtcccgtctctatcgcgacagcctttgccatgctttcattgggaaccaaggccgatacacatgatgaaatcttggaaggtttgaattttaatcttaccgagatcccagaagcccaaatccacgaaggcttccaggaattgctgcgtacgttaaaccaacccgattcacaacttcagttaactaccggaaatgggcttttcttatctgaagggctgaagttggttgataaattcttagaagacgtgaagaaactttatcattcggaggcattcacggtgaacttcggtgacacggaggaagccaaaaagcaaattaacgactatgttgaaaaagggacgcagggtaagatcgtggacttagtaaaggagctggatcgtgataccgtcttcgccttggtaaactacatcttcttcaaaggaaagtgggagcgtccgtttgaggtgaaggatactgaggaggaagatttccatgttgaccaagtgactactgttaaggtccccatgatgaagcgtcttggcatgttcaacatccaacactgcaagaaactgtcgtcatgggtgttgctgatgaaatatcttggtaacgctaccgccattttctttttgcccgatgaaggaaagttacagcaccttgagaacgagcttacccatgatattattacgaaatttttagaaaatgaagaccgtcgttcggcatctttacacttaccgaagcttagtatcactggtacctatgacttgaagtcagttttgggacagcttggcattacgaaggtgttctctaatggagccgacctgtccggcgttacggaggaagcaccattaaagttgagcaaagccgtgcataaagccgttttaactatcgatgaaaaaggaactgaagctgcgggcgcgatgttccttgaggcaattcctatgagcatcccacctgaagttaaattcaataagccttttgtgtttttgatgatcgagcagaacacaaagagtccgttgttcatgggcaaggttgttaaccccacgcagaaataa

SEQ ID NO: 58 - protein, 533 aa, Homo sapiens
  sdAb-linker-AAT(Z=Cys) protein (1)..(533)
MEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGGSEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 59 - DNA, 32 bp, Artificial Sequence
  Forward Primer
  Forward primer for the Asp>Asn mutation at IL15 aa73 position (1)..(32)
gattatcctggctaacaatagcctgtcgagca

SEQ ID NO: 60 - DNA, 32 bp, Artificial Sequence
  Reverse Primer
  Reverse primer for the Asp>Asn mutation at IL15 aa73 position (1)..(32)
tgctcgacaggctattgttagccaggataatc

(19) United States
(12) Patent Application Publication
(10) Pub. No.: US 2021/0253671 A1
(43) Pub. Date: Aug. 19, 2021

(54) Pharmaceutical Composition Containing Fusion Protein and Use Thereof

(71) Applicant: ProtTech Inc., Phoenixville, PA (US)

(72) Inventors: Xiaolong Zhang, Collegeville, PA (US); Yi Zhou Wang, Norristown, PA (US); Huan Shen, Lower Gwynedd, PA (US); Qian Jin, Collegeville, PA (US)

(73) Assignee: ProtTech Inc., Phoenixville, PA (US)

(21) Appl. No.: 16/972,646

(22) PCT Filed: Jun. 7, 2019
(86) PCT No.: PCT/US2019/036175

§ 371 Date: Dec. 7, 2020

Related U.S. Application Data
(60) Provisional application No. 62/682,142, filed on Jun. 7, 2018.

(51) Int. Cl. C07K 14/81; C07K 14/55

(52) U.S. Cl. CPC C07K 14/8125; A61K 38/00; C07K 14/55

(57) ABSTRACT

This disclosure is directed to a fusion protein composition comprising an alpha-1-antitrypsin or α1-antitrypsin (also known as A1AT, A1A, or AAT) polypeptide (AAT), a modified AAT (mAAT) or a functional variant thereof and a bioactive polypeptide. This disclosure is particularly directed to a pharmaceutical composition comprising the fusion protein for treating a disease, such as a cancer or an autoimmune disease. The bioactive polypeptide can be a peptide hormone, interferon, or cytokine, such as interleukin-2 (IL-2), a modified IL-2 (mIL-2), IL-15, G-CSF, GM-CSF, IFN-α2, IFN-β1, GLP-1, FGF21, sdAb, a fragment thereof, a modified polypeptide thereof, or a combination thereof. One advantage of the fusion protein is to enhance the activity, stability, bioavailability or a combination thereof, of the bioactive polypeptide.

### CROSS REFERENCE

This application is a U.S. National-Stage entry under 35 U.S.C. § 371 based on International Application No. PCT/US2019/036175, filed Jun. 7, 2019 which claims the priority of a U.S. provisional application Ser. No. 62/682,142 filed on Jun. 7, 2018, which is hereby incorporated by reference.

### FIELD OF THE INVENTION

This invention is related to fusion proteins comprising a human AAT including a modified AAT (mAAT) polypeptide that can be used as a pharmaceutical composition for delivering a target bioactive agent such as a modified IL-2 for treating human diseases. This invention is further related to a process for producing the fusion protein composition and the pharmaceutical composition.

### BACKGROUND

Proteins and peptides are important biomolecules that have been used in pharmaceutical applications, such as antibodies, antigens, cytokines and hormones, for example, insulin, growth hormones, vaccines, and the like. Modulating or enhancing activities of the proteins or peptides, especially on the delivery and in vivo activities, is of intense research and development.

Cytokines are a group of small proteins that are important in cell signaling. The molecular weight of cytokines is typically in a range of 5-20 KDa. One special feature of the cytokines is that the concentration of cytokines in circulation can vary in a large range. For example, the concentration of IL-6 in blood is typically in picomolar (10⁻¹² M) range. However, it can increase up to 1,000 times during trauma or infection. Interleukins are a group of cytokines that are produced by white blood cells (leukocytes) and many different types of cells including helper CD4 T lymphocytes, monocytes, macrophages, and endothelial cells. The function of the immune system depends mostly on interleukins. They promote the development and differentiation of T and B lymphocytes, and hematopoietic cells.

Interleukin-2 (IL-2) is a member of interleukin family. IL-2 plays an essential role in the basic functions of the immune system. It plays a key role in enduring cell-mediated immunity. When T-cells is stimulated by antigens, IL-2 promotes the differentiation of those T-cells into effector T-cells and memory T-cells clones and promotes the expansion of the antigen-stimulated T-cell clones, thus helping the body to fight off infections and other diseases such as cancer. IL-2 also plays a key role in immune tolerance. IL-2 promotes the differentiation of certain immature T-cells into regulatory T-cells, which can suppress other T-cells and prevent autoimmune diseases.

The IL-2 molecule has the structure of four alpha-helix bundle. The signaling of IL-2 depends on its binding to its receptor, IL-2R, on the surface of T-cells. The IL-2R has three subunits, alpha, beta, and gamma. The gamma chain is shared by all family members in this interleukin group, including IL-4, IL-7, IL-9, IL-15 and IL-21 receptors. IL-2 binds to IL-2R subunit alpha with low affinity. However, the binding of beta and gamma subunits to IL-2R increase the IL-2 binding affinity by about 100-fold. The formation of the IL-2 and 3-subunit IL-2R complex is essential for the transduction of IL-2 signaling in T-cells. IL-2 gene expression is regulated on multiple levels, including the signaling through T-cell receptor (TCR). After the TCR recognizes MHC-peptide complex, a signal is transduced through phospholipase-C (PLC) dependent pathway and activates 3 major transcription factors and their pathways: NFAT, NFkB and AP-1. After co-stimulation from CD28, the IL-2 expression is induced.

Several recombinant IL-2 analogs have been developed and approved for therapeutic applications. For example, Aldesleukin (available from Novartis Vaccines and Diagnostics, Inc. under a registered trademark PROLEUKIN®), originally developed by Cetus Corporation, has the cysteine residue 125 replaced with a serine and the removal of N-terminal alanine. It is approved by the FDA for metastatic renal carcinoma in 1992. Teceleukin, developed by Roche, with a methionine added at protein N-terminal. Bioleukin, developed by Glaxo, also with a methionine added to the protein N-terminal and the cysteine residue 125 replaced with an alanine.

Alpha-1-antitrypsin or α1-antitrypsin (A1AT, A1A, or AAT, hereafter referred to as “AAT”) is a protein belonging to the serpin superfamily. It is also known as alpha1-proteinase inhibitor or alpha1-antiproteinase because it inhibits various proteases. It is encoded in human by the SERPINA1 gene.

The human genome encodes 36 serpin proteins, termed serpinAX through serpinPX (X is a number). Among them, 29 serpin proteins have protease inhibition activity, and 7 serpin proteins do not have protease inhibition activity. Non-inhibitory serpins perform a wide array of important roles. For example, ovalbumin is the most abundant protein in egg white. Although its exact function is unknown, it was speculated to be a storage protein for the developing fetus. Heat shock protein 47 (Hsp 47 also called SERPINH1) is a molecular chaperone that is essential for proper folding of collagen.

Despite their varied functions, all serpins share a common structure. All serpin proteins typically have three β-sheets (named A, B, and C) and eight or nine α-helices (named hA-hl). The most significant regions to serpin function are the A-sheet and the reactive center loop (RCL). The A-sheet includes two β-strands that are in a parallel orientation with a region between them called the “shutter”, and the upper region called a “breach”. The RCL forms the initial interaction with the target protease in inhibitory molecules. All inhibitory serpins use an unusual conformational change to disrupt the protease and to prevent it from completing catalysis. The conformational change involves the RCL moving to the opposite end of the protein and inserting into β-sheet A, forming an extra antiparallel β-strand. This conformational change converts the serpin molecules from a stressed (S) state to a lower-energy relaxed (R) state. This S to R transition is the most notable feature shared by most, if not all, serpin proteins, including non-inhibitory serpins.

AAT is a 52 KDa serpin with a single-chain polypeptide consisting of 394 amino acid residues in its mature form. It exhibits many glycoforms. In adults, AAT protein is produced in the liver and joins the systemic circulation. It has a reference range in the blood of 0.9-2.3 g/L, but the concentration can rise many folds upon acute inflammation. Its main function is to protect tissues from enzymes of inflammatory cells, especially the neutrophil elastase. If the blood contains inadequate amounts of functional AAT protein, such as in AAT deficiency patients, the neutrophil elastase can degrade the elasticity of the lungs and result in respiratory complications, such as chronic obstructive pulmonary disease. For those patients, five AAT products have been approved for therapeutic use, including Aralast NP, Glassia, Prolastin® (a registered trademark of GRIFOLS THERAPEUTICS LLC), Prolastin®-C (a registered trademark of GRIFOLS THERAPEUTICS LLC), and Zemaira® (a registered trademark of CSL BEHRING L.L.C.). Those pharmaceutical forms of AAT are all purified from human donor blood. The recombinant versions are under investigation but are not available yet.

Like all serine protease inhibitors, AAT has a characteristic secondary structure of beta sheets and alpha helices. The primary target of AAT is elastase, but it can also inhibit plasmin and thrombin to some degree. In vitro, AAT can inhibit trypsin (that gives its name “antitrypsin”), chymotrypsin and other serine proteases. Also similar to many other serpins, the mechanism of the protease inhibition involves a large conformational change in AAT structure (the S to R transition). The reactive center loop (RCL) extends out from the body of the AAT protein and directs binding to the target protease. The protease cleaves the serpin at the reactive site within the RCL, establishing a covalent linkage between the carboxyl group of the serpin reactive site and the serine hydroxyl of the protease. The resulting inactive serpin-protease complex is highly stable.

Possibly due to the unique feature of AAT structure, many mutations in AAT can lead to non-functional proteins. Among them, the most notable one is called α1-antitrypsin Pittsburgh (α1-AT-P), initially designated antithrombin Pittsburgh, which was characterized as Met358 to Arg substitution. The Pittsburgh mutation was identified in 1983 in the plasma of a boy who had died at the age of 14 of a severe bleeding disorder. That mutation is located in middle the reactive RCL loop: 344GTEAAGAMFLEAIPMSIPPEVKFNK368 (the numbering here is designated for mature AAT protein without the 24 amino acid signal sequence corresponding to Met382 in its native form). This mutation leads to a potent thrombin inhibition activity.

Although many mutations of AAT are known and can be found at the following website https://www.uniprot.org/uniprot/P01009, new forms of AAT and modified AAT are still needed for improving its utilities and new applications.

### SUMMARY

This invention is directed to a fusion protein composition comprising an AAT polypeptide or a functional variant thereof, and a bioactive polypeptide, wherein the bioactive polypeptide is covalently linked to the AAT polypeptide, covalently linked to said AAT polypeptide via a linker peptide, or a combination thereof. The fusion protein composition comprises a linker peptide that has an N-terminal, a C-terminal and 1-50 amino acid residues and wherein the linker peptide is positioned between said AAT polypeptide and said bioactive polypeptide. The AAT polypeptide can comprise a mAAT polypeptide or a functional variant thereof, wherein the mAAT polypeptide or the functional variant thereof is free from cysteine amino acid residue, wherein the functional variant has at least 85% sequence identity of the mAAT polypeptide and wherein the mAAT polypeptide and the functional variant each is free from serine protease inhibitor activity.

The present invention is also directed to a pharmaceutical composition comprising a fusion protein and, optionally, one or more pharmaceutically acceptable carriers, the fusion protein comprising: an AAT polypeptide or a functional variant thereof; a bioactive polypeptide; wherein, the bioactive polypeptide is covalently linked to said AAT polypeptide, covalently linked to said AAT polypeptide via a linker peptide, or a combination thereof.

The present invention is further directed to an expression vector comprising a coding region comprising AAT codes encoding an AAT polypeptide or a functional variant thereof, and bioactive polypeptide codes encoding a bioactive polypeptide, wherein the AAT codes and the bioactive polypeptide codes are configured to link together directly or via linker codes encoding a linker peptide having an N-terminal, a C-terminal and 1-50 amino acid residues, and wherein the linker codes are positioned between the AAT codes and the bioactive polypeptide codes.

This invention is further directed to process for producing a fusion protein, the process comprising: expressing any one of the expression vectors disclosed herein comprising a coding region encoding the fusion protein in a host to produce a pre-fusion protein; harvesting the pre-fusion protein from cells of the host, cell lysate of the host, an inclusion body of the host, media culturing the host, or a combination thereof; and producing the fusion protein from the pre-fusion protein.

This invention is further directed to a method for treating a disease using the pharmaceutical composition disclosed herein. The disease can be a cancer, an autoimmune disease, diabetes, vasculitis, heart disease, virus infection, or a combination thereof.

### BRIEF DESCRIPTION OF THE FIGURES

FIG. 1. Schematic representations of structures of a fusion protein: (A) a structure having an AAT or mAAT at its C-terminal and (B) a structure having an AAT or mAAT at its N-terminal.

FIG. 2. Examples of mutations of the fusion protein.

FIG. 3. A schematic example of a process for producing a fusion protein.

FIG. 4. A representative example of a gel image of expressed fusion protein IL2-Linker1-AAT(X=Ser, Z=Ser).

FIG. 5. A representative example of a gel image of purified and refolded fusion protein IL2-Linker1-AAT(X=Ser, Z=Ser).

FIG. 6. A representative example of expression, refolding and purification of IL2-Linker1-AAT (X=Ser;Z=Cys) fusion protein with Mw of about 60 kDa. As used herein throughout this disclosure including all Figures: Molecular weight markers (M) are shown in KDa; BI: Before induction; AI: After induction; RF-PU: Refolding and Purification. The fusion protein is indicated with an arrow.

FIG. 7. A representative example of expression, refolding and purification of IL2-Linker2-AAT (X=Ser;Z=Ser) fusion protein with Mw of about 60 kDa.

FIG. 8. A representative example of expression, refolding and purification of IL2-Linker2-AAT (X=Ser;Z=Cys) fusion protein with Mw of about 60 kDa.

FIG. 9. Representative examples of activities of IL2 control (IL2 CN, Solid Diamond), IL2-Linker1-AAT (X=Ser;Z=Ser) (IL2-AAT(S), Solid square) and IL2-Linker1-AAT (X-Ser,Z=Cys) (IL2-AAT(C), Open triangle) measured using CTLL2 cell proliferation assay.

FIG. 10. A representative example of cell stimulation assay. Activities of the IL2 protein Control and a IL2-Linker1-AAT (X=Ser;Z=Ser) fusion protein were measured using CTLL2 cell proliferation assay.

FIG. 11. A representative example of an in vivo tumor inhibition assay. The triple star designates p<0.05.

FIG. 12. A representative assay of Anti-Trypsin function of IL2-Linker2-AAT(X=Ser; Z=Cys) fusion protein. Lanes: M. MW Marker; 1. Antibody PT038 with heavy & light chain; 2. IL2-Linker2-AAT(X=Ser; Z=Cys); 3. Antibody plus trypsin; 4. IL2-Linker2-AAT(X=Ser; Z=Cys) plus trypsin; 5. Antibody and IL2-Linker2-AAT(X=Ser; Z=Cys) plus trypsin; 6. Antibody plus elastase; 7. Antibody and IL2-Linker2-AAT(X=Ser; Z=Cys) plus elastase.

FIG. 13. A representative example of expression, refolding and purification of ID 5-Linker2-AAT (Z=Ser) fusion protein with Mw of about 58 kDa.

FIG. 14. A representative example of expression, refolding and purification of ID 5-Linker2-AAT (Z=Cys) fusion protein with Mw of about 58 kDa.

FIG. 15. Representative examples of activities of fusion proteins. IL15 activities measured using CTLL2 cell proliferation assay: IL2 control (IL2 CN, Solid diamond), m ID 5-Linker2-AAT(X=Asn, Z=Ser) (IL15-AAT(S), Solid square) and mIL15-Linker2-AAT (X=Asn, Z=Cys) (IL15-AAT(C), Open triangle).

FIG. 16. A representative example of expression, refolding and purification of G-CSF-Linker2-AAT (Z=Ser) fusion protein with Mw of about 64 kDa.

FIG. 17. A representative example of expression, refolding and purification of G-CSF-Linker2-AAT (Z=Cys) fusion protein with MW of about 64 kDa.

FIG. 18. Representative examples of G-CSF activities measured using M-NFS-60 cell proliferation assay. G-CSF control: Solid diamond; G-CSF-Linker2-AAT (Z=Ser): G-CSF-AAT(S), Solid square and G-CSF-Linker2-AAT(Z=Cys): G-CSF AAT(C), Open triangle.

FIG. 19. A representative example of expression, refolding and purification of GM-CSF-Linker2-AAT (Z=Ser) fusion protein with Mw of about 60 kDa.

FIG. 20. A representative example of expression, refolding and purification of GM-CSF-Linker2-AAT (Z=Cys) fusion protein with Mw of about 60 kDa.

FIG. 21. Representative examples of GM-CSF activities measured using TF1 cell proliferation assay. GM-CSF control: MG-CSF cn, solid diamond; GM-CSF-Linker2-AAT(Z=Ser): GM-CSF-AAT(S), open triangle; GM-CSF-AAT(Z=Cys): GM-CSF AAT(C), solid square.

FIG. 22. A representative example of expression, refolding and purification of IFNα2-Linker2-AAT (Z=Ser) fusion protein with Mw of about 65 kDa.

FIG. 23. A representative example of expression, refolding and purification of IFNα2-Linker2-AAT (Z=Cys) fusion protein with MW of about 65 kDa.

FIG. 24. A representative example of expression, refolding and purification of IFNβ1-Linker2-AAT (Z=Ser) fusion protein with Mw of about 65 kDa.

FIG. 25. A representative example of expression, refolding and purification of IFNβ1-Linker2-AAT (Z=Cys) fusion protein with Mw of about 65 kDa.

FIG. 26. A representative example of expression, refolding and purification of GLP1-Linker2-AAT (Z=Ser) fusion protein with Mw of about 48 kDa.

FIG. 27. A representative example of expression, refolding and purification of GLP1-Linker2-AAT (Z=Cys) fusion protein with Mw of about 48 kDa.

FIG. 28. A representative example of expression, refolding and purification of AAT(Z=Ser)-Linker2-FGF21 fusion protein with Mw of about 65 kDa.

FIG. 29. A representative example of expression, refolding and purification of AAT(Z=Cys)-Linker2-FGF21 fusion protein with Mw of about 65 kDa.

FIG. 30. A representative example of expression, refolding and purification of sdAb-Linker2-AAT (Z=Ser) fusion protein with Mw of about 59 kDa.

FIG. 31. A representative example of expression, refolding and purification of sdAb-Linker2-AAT (Z=Cys) fusion protein with Mw of about 59 kDa.

FIG. 32. Representative examples of trypsin protease inhibition function assay for fusion proteins GLP1-Linker2-AAT(Z=Cys), AAT(Z=Cys)-Linker2-FGF21, G-CSF-Linker2-AAT(Z=Ser) and GM-CS F-Linker2-AAT(Z=Cys). Lanes: 1. GLP1-Linker2-AAT(Z=Cys); 2. GLP1-Linker2-AAT(Z=Cys) plus trypsin; 3. AAT(Z=Cys)-Linker2-FGF21; 4. AAT(Z=Cys)-Linker2-FGF21 plus trypsin; 5. G-CSF-Linker2-AAT(Z=Ser); 6. G-CSF-Linker2-AAT(Z=Ser) plus trypsin; 7. G-CSF-Linker2-AAT(Z=Cys); 8. G-CSF-Linker2-AAT(Z=Cys) plus trypsin; 9. GM-CSF-Linker2-AAT(Z=Ser); 10. GM-CSF-Linker2-AAT(Z=Ser) plus trypsin; 11. GM-CSF-Linker2-AAT(Z=Cys); 12. GM-CSF-Linker2-AAT(Z=Cys) plus trypsin.

### DETAILED DESCRIPTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein,

The term “protein”, “proteins”, “peptide”, “peptides”, “polypeptide” or “polypeptide” refers to one or more biomolecules each having a chain of amino acid residues, modified amino acid residues, or a combination thereof. The terms may be used interchangeably throughout this disclosure unless specifically defined otherwise. For example, the term “bioactive polypeptide” can also include “bioactive peptide” or “bioactive protein”. The term can refer to natural biomolecules or synthetic molecules including molecule synthesized via chemical synthesis or produced via a biosystem such as an expression vector and host cells or a cell-free system.

The term “bioactive agent” or a grammatical variant refers to a natural or a synthetic material, a compound, a molecule, a part thereof, or a combination thereof that can have biological activity in vivo or in vitro. A bioactive agent can be a large molecule, such as a protein, a peptide, a polypeptide, an antibody, a monoclonal antibody, a derivative or a fragment of an antibody, a nucleotide, a polynucleotide, such as an oligonucleotide, a DNA, an RNA, a small molecule, such as a compound, an aggregate of one or more molecules, a complex of multiple molecules or substances, or a combination thereof. A bioactive agent can include bioactive polypeptide.

The term “fusion protein”, “fusion proteins”, “fusion peptide”, “fusion polypeptide”, “fusion polypeptides”, “chimeric protein” or “chimeric polypeptide” refers a biomolecule having a chain of amino acid residues that have identity of similarity to two or more proteins or fragments thereof.

The term “AAT”, “A1AT” or “A1A” refers to Alpha-1-antitrypsin, α1-antitrypsin, alpha1-proteinase inhibitor or alpha1-antiproteinase, collectively referred to as “AAT”. The AAT is encoded in human by the SERPINA1 gene. The term “AAT” also includes modified AAT (mAAT). Throughout The term “mAAT” refers to a modified AAT. The modification can comprise at least one amino acid mutation or modification at at least one position of the AAT polypeptide, addition or truncation of one or more amino acids at the N-terminal of the AAT, addition or truncation of one or more amino acids at the C-terminal of the AAT, or a combination thereof. The term “mAAT” can also refer to a modified AAT coding sequence. In examples, a mAAT can have a mutation at a particular position, such as at the Z position as disclosed herein. In other examples, an AAT can have a truncated or deleted signal peptide (also referred to as a signal sequence) or have one or more additional amino acids. In further examples, the term AAT or mAAT can also refer to a cDNA sequence that comprise codons optimized for expression in a certain host, such as codons optimized for expression in E. coli host. The modifications disclosed above or hereafter can be suitable. Generally, when an AAT is modified with another protein, the modified AAT protein can also be referred to as a fusion protein.

This invention is directed to a fusion protein composition comprising an AAT polypeptide or a functional variant thereof, and a bioactive polypeptide, wherein the bioactive polypeptide is covalently linked to the AAT polypeptide, covalently linked to the AAT polypeptide via a linker peptide, or a combination thereof.

The fusion protein composition can comprise a linker peptide that has an N-terminal, a C-terminal and 1-50 amino acid residues and wherein the linker peptide is positioned between the AAT polypeptide and the bioactive polypeptide.

In embodiments, the bioactive polypeptide can be linked to the N-terminal of the linker peptide and the AAT polypeptide can be linked to the C-terminal of the linker peptide.

In other embodiments, the bioactive polypeptide can be linked to the C-terminal of the linker peptide and the AAT polypeptide can be linked to the N-terminal of the linker peptide.

The fusion protein composition of this invention can comprise a mAAT (modified AAT) polypeptide or a functional variant thereof, wherein said mAAT polypeptide is free from cysteine (herein referred to as Cys or C) amino acid residue, wherein the functional variant has at least 85% sequence identity of the mAAT polypeptide and wherein the mAAT polypeptide and the functional variant each is free from serine protease inhibitor activity. Percentage is based on the number of amino acid residues in the mAAT.

Different from the original human AAT, a mAAT can comprise a mutation at the Z position (defined hereafter) where the original cysteine (C) is replace by another amino acid different from cysteine. In one example, the mAAT can have an amino acid sequence identified by SEQ ID. 1 where the original cysteine (C) is replace by a serine (S) (the Z position in SEQ ID. 1 is amino acid position 232). In another example, the amino acid at the Z position can be selected from Z=A, R, N, D, E, Q, G, H, I, L, K, M, F, P, S, T, W, Y or V. In a further example, the amino acid at the Z position can be selected from Z=S or A. The original human AAT without its signal sequence is shown as SEQ ID. 2 with its original cysteine at the Z position. A full polypeptide sequence of the original human AAT including the signal sequence is shown as SEQ ID. 3. The mAAT can be a synthesized polypeptide with one or more mutations, wherein at least one of the mutations is at the Z position having an amino acid selected from Z=A, R, N, D, B, E, Q, G, H, I, L, K, M, F, P, S, T, W, Y or V. In examples, a fusion protein composition of this invention can comprise a mAAT having a serine or an alanine mutation at a Z position in the mAAT.

The fusion protein composition can comprise further amino acid residues or polypeptides linked to the mAAT polypeptide or the functional variant thereof. In one example, the protein composition can comprise a mAAT polypeptide with additional methionine (M) linked to its N-terminal, a signal peptide linked to its N-terminal, or other amino acid, peptide or polypeptide linked to it N-terminal or C-terminal.

The functional variant of the mAAT can have at least 85% sequence identity of the mAAT polypeptide, based on the number of amino acid residues of the mAAT. The functional variant thereof can have in a range of from 85% to 100% identify of the mAAT polypeptide in one example, 90% to 100% identify in another example, 95% to 100% identify in yet another example and 98% to 100% identify in a further example, based on the number of amino acid residues of mAAT. The functional variant of the mAAT can be free from cysteine (C) and can have amino acid at the Z position selected from Z=A, R, N, D, E, Q, G, H, I, L, K, M, F, P, S, T, W, Y or V. In particular examples, the functional variant of the mAAT can have amino acid at the Z position selected from Z=S or Z=A.

The mAAT polypeptide and the functional variant each is free from serine protease inhibitor activity.

The fusion protein composition disclosed herein can comprise a bioactive polypeptide covalently linked to the AAT or mAAT polypeptide or covalently linked to the mAAT polypeptide via a linker peptide. The bioactive polypeptide can be directly covalently linked to the mAAT polypeptide without a linker peptide in one example or covalently linked to the mAAT polypeptide via a linker peptide in another example. In a further example, the fusion protein composition can comprise a fusion protein comprising a mAAT (modified AAT) polypeptide or a functional variant thereof and a bioactive polypeptide covalently linked to the mAAT polypeptide or covalently linked to the mAAT polypeptide via a linker peptide.

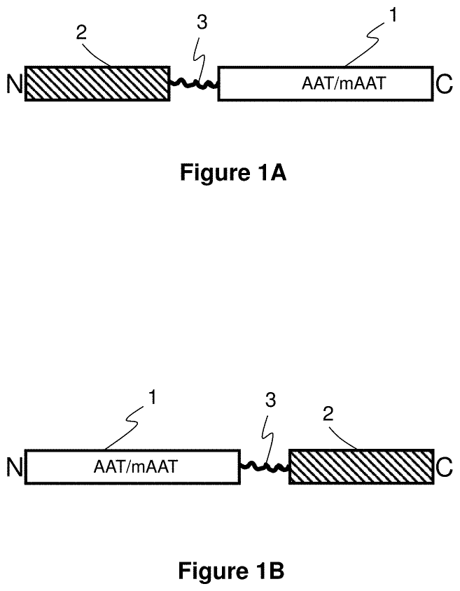

Schematic representations of fusion proteins are shown in FIG. 1A and FIG. 1B with an AAT or mAAT polypeptide (1) linked to a bioactive polypeptide (2) via a linker peptide (3). The fusion proteins are shown with the N-terminal (N), also known as NH₂-terminal or amine-terminal to the left and the C-terminal (C), also known as carboxyl-terminal, carboxy-terminal, C-terminal tail, C-terminal end, or COOH-terminal to the right.

The linker peptide can have an N-terminal, a C-terminal and 1-50 amino acid residues and wherein the linker peptide is positioned between the AAT or the mAAT polypeptide and the bioactive polypeptide (FIG. 1A-FIG. 1B). In one example, the bioactive polypeptide is linked to the N-terminal of the linker peptide and the AAT or mAAT polypeptide is linked to the C-terminal of the linker peptide (FIG. 1A). In another example, the bioactive polypeptide is linked to the C-terminal of the linker peptide and the AAT or the mAAT polypeptide is linked to the N-terminal of the linker peptide (FIG. 1B). The first Met (M) residue can be optional for the fusion protein. In one example, a first M can be encoded in a fusion protein coding region and expressed in a host. In another example, the first M can be subsequently removed from the fusion protein in the host cells, such as by aminopeptidases.

The linker peptide can have in a range of from 1 to 50 amino acid residues. When present, the linker peptide can affect the expression yield, structure, contribute to the stability, activity, bioavailability and in vivo metabolism of a fusion protein. In general, although many different linker peptide sequences may be used satisfactorily for a given fusion protein, the suitability of a linker sequence in the fusion protein has to be determined experimentally.

Fusion protein linkers are generally classified into 3 categories according to their structures: flexible linkers, rigid linkers and in vivo cleavable linkers. All three types of linker have been used successfully in making functional fusion proteins.

The linker peptide can be a flexible linker when two joining protein domains need a certain degree of freedom in their movement and interaction with other proteins. A flexible linker can consist of small amino acid residues such as glycine, serine or a combination thereof. Thr and Ala can also be added to modify its flexibility. As the smallest amino acid in size, glycine can provide a high degree of flexibility. Serine or Thr can help to maintain the stability of a linker in aqueous solution by forming hydrogen bonds with water molecules and reduces the unfavorable interactions between the protein domains or moieties and the linker. Examples of suitable flexible linkers can include (Gly)₆, (Gly)₈, (Gly-Gly-Gly-Gly-Ser)ₙ (n=1, 2 or 4) (also known as a (G₄S)ₙ linker), or variants thereof. Many other similar linker sequences, such as incorporating Thr or Ala into a (G₄S)ₙ linker, can also be suitable to provide similar functionality as a flexible linker.

The linker peptide can be a rigid linker peptide, for example, when a fusion protein with a flexible linker has some expression or activity issues, or a spatial separation of joining protein domains is required. A rigid linker peptide can maintain the distance between the protein domains in a fusion protein. Two forms of rigid linkers can be suitable: such as a (EAAAK)ₙ linker, which has an E to K salt bridge and forms a helical structure; or a (XP)ₙ linker, in which X can be any amino acid, preferably Ala, Lys or Glu. The presence of multiple Proline residues in a linker peptide can increase its stiffness and spatial separation between two joining protein domains.

Both flexible and rigid linkers are stable in vivo and do not allow the separation of joined proteins or protein domains. A cleavable linker, on the other hand, permits the separation of joining proteins or protein domains releasing a free protein domain in vivo. The cleavable linker peptide can comprise one or more disulfide bonds or one or more proteolytic cleavable peptide bonds. Reduction of the disulfide bond or proteolytic cleavage can result in the separation of the joining protein domains. Cleavable linkers can be utilized to improve the bioactivity or targeting a protein drug to a specific tissue or cells. Examples of the cleavable linkers include cyclopeptide linkers, which contain a disulfide linkage between two Cys residues, and protease-sensitive linkers, which contain a cleavage site sensitive to proteases present in specific tissues or intracellular compartments, such as matrix metalloproteinases (MMPs), furin encoded by the FURIN gene (also known as PACE, Paired basic Amino acid Cleaving Enzyme) and cathepsin B, a lysosomal cysteine protease.

A linker peptide Suitable to this invention can be a flexible linker. A rigid linker can also be suitable depending on molecular structures the AAT or mAAT polypeptide and the bioactive polypeptide. A linker peptide can comprise small amino acid residues such as a GSTSGS peptide (SEQ ID. 15) in one example or a modified (G₄S)ₙ linker such as a GGGGSGGGGS peptide (SEQ ID. 16) in another example.

In fusion protein composition disclosed herein, the bioactive polypeptide can comprise a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof. The term “antibody” used herein can include a polyclonal antibody (Ab), a monoclonal antibody (mAb), a tri-functional mab, a bifunctional mAb, a cross mAb, an IgG, an IgM, a DV_Ig, an IgG-scFV, scFv2-Fc, Bi-Nanobody, BiTE, tandABs, or DART. The bioactive polypeptide can have a molecular weight in a range of from 100 to 500,000 Daltons (or 0.1 to 500 KDa). The bioactive polypeptide can have a molecular weight in a range of from 100 to 500,000 Daltons in one example, 100 to 250,000 Daltons in another example, 100 to 150,000 Daltons in yet another example, 100 to 100,000 Daltons in yet another example, 100 to 75,000 Daltons in yet another example, 100 to 50,000 Daltons in yet another example and 100 to 25,000 in yet a further example. In preferred examples, bioactive polypeptide can have a molecular weight in a range of from 100 to 25,000 Daltons. In yet further examples, the bioactive polypeptide can have a molecular weight in a range of from 100 to 24,000 Daltons. In yet further examples, the bioactive polypeptide can have 0 to 3 disulfide bonds. For a protein expressed in E. coli, it can be difficult to refold when there are more than 3 disulfide bonds within the protein or between protein molecules, for example, when disulfide bonds formed between one amino acid of one protein polypeptide and another amino acid of another protein polypeptide. In yet further examples, the bioactive polypeptide can have a molecular weight in a range of from 100 to 24,000 Daltons, 0 to 3 disulfide bonds or a combination thereof.

The bioactive polypeptide can comprise one or more neoantigens or epitopes. In one example, mutant MHC class II epitopes identified by Kreiter et al. (NATURE, 692, VOL 520, 30 Apr. 2015) can be suitable as bioactive polypeptides for driving therapeutic immune responses in cancer patients.

In examples, the bioactive polypeptide can comprise one or more interferons (IFNs), such as Type I, II or III INFs. The bioactive polypeptide can comprise mammalian type I IFNs including IFN-α, IFN-β, IFN-δ, IFN-ε, IFN-κ, IFN-ω, IFN-μ, IFN-τ or IFN-ζ in one example, Type II IFN-γ in another example, and Type III interferons including IFN-λ1 (IL-29), IFN-λ2 (IL-28A), and IFN-λ3 (IL-28B) in a further example. In further examples, the bioactive polypeptide can comprise Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof. The term “a fragment thereof” used herein refers to a fragment of a polypeptide disclosed herein. The term “modified polypeptide” refers to a polypeptide comprises at least one mutation, deletion, addition, or a combination thereof, such as a mutation that changes at least one amino acid residue at at least one position. In one example, Asn72 of human ID 5 can be mutated to Asp72. In another example, Cys17 of human G-CSF can be mutated to Ser17. In yet another example, Ala2 of human GLP1 (7-37) peptide can be changed to Gly2. In yet another example, Cys 16 in human IFN-βb1 can be changed to Ser 16. The numbering used herein can be based on a sequence without signal sequence or the Met residue at the N-terminal.

Suitable to the fusion protein composition of this invention, the bioactive polypeptide can comprise an interleukin-2 (IL-2) or a modified IL-2 (mIL-2). In examples, the interleukin-2 (IL-2) can be a human IL-2, such as the one identified in SEQ ID. 4. The modified IL-2 (mIL-2) can be a modified human IL-2, such as those identified in SEQ ID. 10 and SEQ ID 11. The modified IL-2 can comprise a serine or an alanine mutation at an X position in the mIL-2, i.e., a mutation that replaces a Cys at a X position with a Ser or an Ala. The fusion protein composition can comprise a mAAT polypeptide and a mIL-2 polypeptide linked together with a linker peptide having a serine or an alanine mutation at X position in the mIL-2 polypeptide and a serine or an alanine mutation at Z position in the mAAT polypeptide.

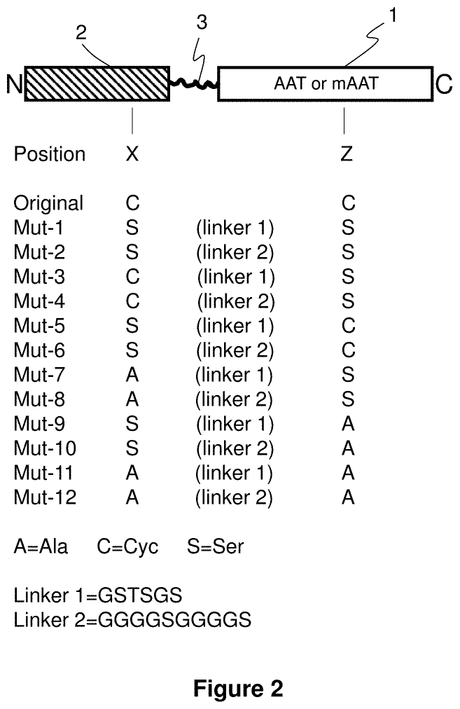

The X position is defined as the amino acid position 125 that is a cysteine (125Cys) in the original human IL-2 polypeptide without signal sequence (145Cys when the 20 amino acid signal sequence is considered) regardless of actual amino acid position number in a fusion protein that may shift due to variations in leading sequence such as signal sequence, removal or addition of the first methionine residue, lengths of linkers, or any other variations. For example, when X=C, the amino acid at the position 125 of an IL-2 is a cysteine and when X=S, the amino acid at the position 125 of a mIL-2 is a serine, and so on. The term “Z position” or grammatical variant used herein throughout this disclosure is defined as the amino acid position 256 that is a cysteine (256Cys) in the original human AAT polypeptide with signal sequence (232Cys when the 24 amino acid signal sequence is absent) regardless of actual amino acid position number in a fusion protein that may shift due to variations in leading sequence such as signal sequence, removal or addition of the first methionine residue, length of linker, or any other variations. For example, when Z=C, the amino acid at the position 256 of an AAT is a cysteine (C) and when Z=S, the amino acid at the position 256 of a mAAT is a serine (S), and so on. In one example, X=A, R, N, D, C, E, Q, G, H, I, L, K, M, F, P, S, T, W, Y or V. In another example, Z=A, R, N, D, C, E, Q, G, H, I, L, K, M, F, P, S, T, W, Y or V. In a further example, X=S or A, Z=S or A, or a combination thereof. The X and Z positions and corresponding mutations are schematically illustrated in FIG. 2. Examples of fusion proteins and combinations Fusion 1 through Fusion 14 are shown in Table 1. Each of the fusion proteins 3-14 comprises a mAAT polypeptide with a specified amino residue at the Z position, a mIL-2 polypeptide with a specified amino residue at the X position and a linker peptide specified. The fusion proteins 1-2 each comprises an AAT polypeptide with an original Cys residue at the Z position, an IL-2 polypeptide with an original Cys residue at the X position and a linker peptide specified.

In one example, a fusion protein can comprise a mIL-2 polypeptide with X=S, a short linker and a mAAT polypeptide with Z=S (SEQ ID. 5). In another example, a fusion protein can comprise a mIL-2 polypeptide with X=S, a long linker and a mAAT polypeptide with Z=S (SEQ ID. 6).

TABLE 1
Examples of Fusion Proteins Comprising
AAT (mAAT) and IL-2 (mIL-2) Combinations
(only the amino acid residues flanking
the X or the Z positions are shown).

| | X Position | | Z Position |
|---|---|---|---|
| | IL-2 (125) | | mAAT (256) |
| Fusion | RWITFXQ | | NIQHZK |
| Protein | SIISTLT | Linker | KLSSWVL |
| Fusion 1 | SEQ ID. 9 X = C | GSTSGS | SEQ ID. 12 |
| | | | Z = C |
| Fusion 2 | SEQ ID. 9 X = C | GGGGSGG | SEQ ID. 12 |
| | | GGS | Z = C |
| Fusion 3 | SEQ ID. 10 X = S | GSTSGS | SEQ ID. 13 |
| | | | Z = S |
| Fusion 4 | SEQ ID. 10 X = S | GGGGSGG | SEQ ID. 13 |
| | | GGS | Z = S |
| Fusion 5 | SEQ ID. 9 X = C | GSTSGS | SEQ ID. 13 |
| | | | Z = S |
| Fusion 6 | SEQ ID. 9 X = C | GGGGSGG | SEQ ID. 13 |
| | | GGS | Z = S |
| Fusion 7 | SEQ ID. 10 X = S | GSTSGS | SEQ ID. 12 |
| | | | Z = C |
| Fusion 8 | SEQ ID. 10 X = S | GGGGSGG | SEQ ID. 12 |
| | | GGS | Z = C |
| Fusion 9 | SEQ ID. 11 X = A | GSTSGS | SEQ ID. 13 |
| | | | Z = S |
| Fusion 10 | SEQ ID. 11 X = A | GGGGSGG | SEQ ID. 13 |
| | | GGS | Z = S |
| Fusion 11 | SEQ ID. 10 X = S | GSTSGS | SEQ ID. 14 |
| | | | Z = A |
| Fusion 12 | SEQ ID. 10 X = S | GGGGSGG | SEQ ID. 14 |
| | | GGS | Z = A |
| Fusion 13 | SEQ ID. 11 X = A | GSTSGS | SEQ ID. 14 |
| | | | Z = A |
| Fusion 14 | SEQ ID. 11 X = A | GGGGSGG | SEQ ID. 14 |
| | | GGS | Z = A |

The X position in other bioactive polypeptides may vary depending on each individual polypeptide if a mutation at such a position is desired. For example, in ID 5, the X position is defined as amino acid 73 of the original ID 5 polypeptide. In most cases, the first Met of a bioactive polypeptide, including many of the bioactive polypeptides disclosed herein, may be removed by E. coli methionine amino peptidase.

Suitable to the fusion protein composition of this invention, bioactive polypeptide can comprise an interleukin-15 (IL-15) or a modified IL-15 (mIL-15). In examples, a fusion protein can comprise bioactive polypeptide comprising cytokine mIL-15 polypeptide with amino acid position 73 replaced with an Asp (X=Asp) (the sequence of the region is 64-VENLIILANDSLSSNGN-80) linked to a long linker (Linker2) and a mAAT polypeptide with Z=Ser (herein referred to as mIL15(X=Asp, Z=Ser), SEQ ID. 26). A fusion protein comprising the aforementioned mIL15 (X=Asp, Z=Ser) can be expressed as soluble protein in E. coli BL21 cells. In other examples, a fusion protein can comprise a bioactive polypeptide comprising a mIL-15 polypeptide with amino acid position 73 replace with an Asn (the sequence of the region is 64-VENLIILANNSLSSNGN-80) linked to a long linker (Linker2) and a mAAT polypeptide with Z=Ser (herein referred to as mIL15(X=Asn, Z=Ser), SEQ ID. 28). A fusion protein comprising the aforementioned mIL15 (X=Asn, Z=Ser) can be expressed mainly as inclusion body in E. coli BL21 cells and can be converted to a biologically active fusion protein via a refolding procedure as disclosed herein. In yet other examples, a fusion protein can comprise a bioactive polypeptide comprising a mIL-15 polypeptide with amino acid position 73 replace with an Asn (the sequence of the region is 64-VENLIILANNSLSSNGN-80) linked to a long linker (Linker2) and a mAAT polypeptide with Z=Cys (herein referred to as mIL15(X=Asn, Z=Cys), SEQ ID. 30). A fusion protein comprising the aforementioned mIL15 (X=Asn, Z=Cys) can be expressed mainly as inclusion body in E. coli BL21 cells and can be converted to a biologically active fusion protein via a refolding procedure as disclosed herein. The IL15 or mIL15 can also comprise a cDNA sequence that comprises modified codons optimized for expression in a host, such as in E. coli host.

Suitable to the fusion protein composition of this invention, the bioactive polypeptide can comprise a G-CSF or a modified G-CSF (mG-CSF). In one example, a fusion protein can comprise a bioactive polypeptide comprising a cell growth factor (G-CSF) linked to a linker peptide and a mAAT polypeptide with Z=Ser (G-CSF-Linker-mAAT(Z=Ser), SEQ ID. 32). In another example, a fusion protein can comprise a bioactive polypeptide comprising a cell growth factor (G-CSF) linked to a linker peptide and mAAT polypeptide with Z=Cys (G-CSF-Linker-mAAT(Z=Cys), SEQ ID. 34). Both fusion proteins can be expressed in E. coli BL21 cells as inclusion bodies at high expression level. Both fusion proteins can be refolded with a high yield and can have biological activity of native G-CSF. Any G-CSF that has one or more mutations and retains some or all of the native G-CSF activity can be suitable as an mG-CSF. In one example, a mG-CSF can comprise a C18S mutation, i.e., an original Cys is mutated to a Ser at the amino acid position 18 (X position for G-CSF) of the G-CSF polypeptide. A mG-CSF can also comprise a cDNA sequence that comprises modified codons optimized for expression in a host, such as in E. coli host.

Suitable to the fusion protein composition of this invention, the bioactive polypeptide can comprise a GM-CSF or a modified GM-CSF (mGM-CSF). In one example, a fusion protein can comprise the bioactive polypeptide comprising a cell growth factor granulocyte-macrophage colony-stimulating factor (GM-CSF) linked to a linker peptide and a mAAT polypeptide with Z=Ser (GM-CSF-Linker-AAT(Z=Ser), SEQ ID. 36). In another example, a fusion protein can comprise a bioactive polypeptide comprising GM-CSF linked to a linker peptide and a mAAT polypeptide with Z=Cys (GM-CSF-Linker-AAT(Z=Cys), SEQ ID. 38). Both fusion proteins can be expressed in E. coli BL21 cells as inclusion bodies at high expression level. Both fusion proteins can be refolded with a high yield and had biological activity of native GM-CSF. A mGM-CSF can also comprise a cDNA sequence that comprises modified codons optimized for expression in a host, such as in E. coli host.

The bioactive polypeptide can comprise IFN-α2 or a modified IFN-α2 (mIFN-α2). In one example, a fusion protein can comprise a bioactive polypeptide comprising IFN-α2 linked to a linker peptide and mAAT polypeptide with Z=Ser (IFN-α2-Linker-AAT(Z=Ser), SEQ ID. 40). In another example, a fusion protein can comprise a bioactive polypeptide comprising IFN-α2 linked to a linker peptide and a mAAT polypeptide with Z=Cys (IFN-α2-Linker-AAT(Z=Cys), SEQ ID. 42). Both fusion proteins can be expressed in E. coli BL21 cells as inclusion bodies at high expression level. Both fusion proteins can be refolded with a high yield. Any IFN-α2 that has one or more mutations and retains some or all of the native IFN-α2 activity can be suitable as a mINF-α2. A mINF-α2 can also comprise a cDNA sequence that comprises modified codons optimized for expression in a host, such as in E. coli host.

The bioactive polypeptide can comprise IFN-β1 or a modified IFN-β1 (mIFN-β1). In one example, a fusion protein can comprise a bioactive polypeptide comprising IFN-β1 linked to a linker peptide and mAAT polypeptide with Z=Ser (IFN-β1-Linker-AAT(Z=Ser), SEQ ID. 44). In another example, a fusion protein can comprise a bioactive polypeptide comprising IFN-β1 linked to a linker peptide and a mAAT polypeptide with Z=Cys (IFN-β1-Linker-AAT(Z=Cys), SEQ ID. 46). Both fusion proteins can be expressed in E. coli BL21 cells as inclusion bodies at high expression level. Both fusion proteins can be refolded with a high yield. Any IFN-β1 that has one or more mutations and retains some or all of the native IFN-β1 activity can be suitable as a mIFN-β1. A mINF-β1 can also comprise a cDNA sequence that comprises modified codons optimized for expression in a host, such as in E. coli host. In yet an example, a bioactive polypeptide comprising IFN-β1 having a C17S mutation, i.e., an original Cys is mutated to a Ser at the amino acid position 17 (X position for IFN-β1) of the IFN-β1 polypeptide.

The bioactive polypeptide can comprise GLP-1 or a modified GLP-1 (mGLP-1). In one example, a fusion protein can comprise a bioactive polypeptide comprising a peptide hormone analog mGLP-1 linked to a linker peptide and an AAT polypeptide with Z=Ser (GLP-1-Linker-AAT(Z=Ser), SEQ ID. 48). In another example, a fusion protein can comprise a bioactive polypeptide comprising a peptide hormone analog mGLP-1 linked to a linker peptide and an AAT polypeptide with Z=Cys (GLP-1-Linker-AAT(Z=Cys), SEQ ID. 50). Both fusion proteins can be expressed in E. coli BL21 cells as inclusion bodies at high expression level. Both fusion proteins can be refolded with a high yield. Any GLP-1 that has one or more mutations and retains some or all of the native GLP-1 activity can be suitable as a mGLP-1. A mGLP-1 can also comprise a cDNA sequence that comprises modified codons optimized for expression in a host, such as in E. coli host. In yet an example, a bioactive polypeptide can comprise a GLP-1 having an A2G mutation, i.e., an original Ala is mutated to a Gly at the amino acid position 2 (X position for GLP-1) of the GLP-1 polypeptide.

The bioactive polypeptide can comprise FGF21 or a modified FGF21 (mFGF21). In one example, a fusion protein can comprise a bioactive polypeptide comprising a cell growth factor, FGF21, fused to the C-terminal of mAAT polypeptide via a linker peptide and with Z=Ser in the mAAT (AAT(Z=Ser)-Linker-FGF21, SEQ ID. 52). In another example, a fusion protein can comprise a bioactive polypeptide comprising FGF21 fused to the C-terminal of mAAT polypeptide via a linker peptide and with Z=Cys in the mAAT (AAT(Z=Cys)-Linker-FGF21, SEQ ID. 54). Both fusion proteins can be expressed in E. coli BL21 cells as inclusion bodies at high expression level. Both fusion proteins can be refolded with a high yield. Any FGF21 that has one or more mutations and retains some or all of the native FGF21 activity can be suitable as a mFGF21. A mFGF21 can also comprise a cDNA sequence that comprises modified codons optimized for expression in a host, such as in E. coli host. In yet an example, a bioactive polypeptide can comprise a FGF21 having a truncation at its C-terminal.

The bioactive polypeptide can comprise sdAb or a modified sdAb (msdAb). In one example, a fusion protein can comprise a bioactive polypeptide comprising a single domain antibody (sdAb) linked to a linker peptide and a mAAT polypeptide with Z=Ser (sdAb-Linker-AAT(Z=Ser), SEQ ID. 56). In another example, a fusion protein can comprise a bioactive polypeptide comprising a single domain antibody linked to a linker peptide and a mAAT polypeptide with Z=Cys (sdAb-Linker-AAT(Z=Cys), SEQ ID. 58). Both fusion proteins can be expressed in E. coli BL21 cells as inclusion bodies at high expression level. Both fusion proteins can be refolded with a high yield. Any sdAb that has one or more mutations and retains some or all of the native sdAb activity can be suitable as a msdAb. An msdAb can also comprise a cDNA sequence that comprises modified codons optimized for expression in a host, such as in E. coli host.

The protein composition disclosed herein can further comprise a targeting agent covalently linked to the AAT or mAAT polypeptide, the bioactive polypeptide, or a combination thereof.

The targeting agent can comprise an antibody, an antibody fragment, antigen, neoantigen or a combination thereof. The targeting agent can be used to target the fusion protein to a specific location in a bio-subject, such as a patient. A targeting agent can be covalently linked to mAAT of a fusion protein in one example, to the bioactive polypeptide of the fusion protein in another example, or both the mAAT and the bioactive polypeptide of the fusion protein in yet another example. A targeting agent can be covalently linked to mAAT of a mAAT-mIL-2 fusion protein in one example, to mIL-2 of the fusion protein in another example, or both the mAAT and IL-2 of the fusion protein in yet another example.

This invention is also directed to a pharmaceutical composition comprising a fusion protein and, optionally, one or more pharmaceutically acceptable carriers, the fusion protein comprising:

an AAT polypeptide or a functional variant thereof;

a bioactive polypeptide;

wherein, the bioactive polypeptide is covalently linked to the AAT polypeptide, covalently linked to the AAT polypeptide via a linker peptide, or a combination thereof.

Any of the aforementioned fusion proteins can be Suitable to the pharmaceutical composition. The fusion protein can comprises a linker peptide that has an N-terminal, a C-terminal and 1-50 amino acid residues and wherein the linker peptide is positioned between the AAT polypeptide and the bioactive polypeptide. The aforementioned linker peptides can be suitable.

In one example, the bioactive polypeptide can be covalently linked to the N-terminal of the linker peptide and the AAT polypeptide can be covalently linked to the C-terminal of the linker peptide. In another example, the bioactive polypeptide can be linked to the C-terminal of the linker peptide and the AAT polypeptide can be linked to the N-terminal of the linker peptide.

Suitable to the pharmaceutical composition of this invention, the AAT polypeptide can comprise a mAAT polypeptide or a functional variant thereof, wherein the mAAT polypeptide or the functional variant thereof can be free from cysteine amino acid residue, wherein the functional variant can have at least 85% sequence identity of the mAAT polypeptide and wherein the mAAT polypeptide and the functional variant each is free from serine protease inhibitor activity.

Suitable to the pharmaceutical composition of this invention, the fusion protein can comprise a mAAT having a serine or an alanine mutation at a Z position in the mAAT.

Suitable to the pharmaceutical composition disclosed herein, the bioactive polypeptide can comprise a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof. Any of the aforementioned bioactive polypeptide can be suitable. The bioactive polypeptide can have a molecular weight in a range of from 100 to 500,000 Daltons in one example, 100 to 250,000 Daltons in another example, 100 to 150,000 Daltons in yet another example, 100 to 100,000 Daltons in yet another example, 100 to 75,000 Daltons in yet another example, 100 to 50,000 Daltons in yet another example and 100 to 25,000 in yet a further example. In one particular example, a bioactive polypeptide can have a molecular weight in a range of from 100 to 25,000 Daltons. In yet further examples, the bioactive polypeptide can have a molecular weight in a range of from 100 to 24,000 Daltons. In yet further examples, the bioactive polypeptide can have 0 to 3 disulfide bonds. In yet further examples, the bioactive polypeptide can have a molecular weight in a range of from 100 to 24,000 Daltons, 0 to 3 disulfide bonds or a combination thereof.

Suitable to the pharmaceutical composition of this invention, a bioactive polypeptide can comprise Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

The bioactive polypeptide can comprise an interleukin-2 (IL-2) in one example or a modified IL-2 (mIL-2) in another example. The mIL-2 can comprise a serine or an alanine mutation at an X position in the mIL-2. In the pharmaceutical composition disclosed herein, the fusion protein can comprise a mAAT and a mIL-2 having a serine or an alanine mutation at a X position in the mIL-2 and a serine or an alanine mutation at a Z position in the mAAT.

Suitable to the pharmaceutical composition of this invention, the bioactive polypeptide can comprise an interleukin-15 (IL-15) or a modified IL-15 (mIL-15), as disclosed above.

Suitable to the pharmaceutical composition of this invention, the bioactive polypeptide can comprise a G-CSF or a modified G-CSF (mG-CSF), as disclosed above.

Suitable to the pharmaceutical composition of this invention, the bioactive polypeptide can comprise IFN-α2 or a modified IFN-α2 (mIFN-α2), as disclosed above.

Suitable to the pharmaceutical composition of this invention, the bioactive polypeptide can comprise IFN-β1 or a modified IFN-β1 (mIFN-β1), as disclosed above.

Suitable to the pharmaceutical composition of this invention, the bioactive polypeptide can comprise GLP-1 or a modified GLP-1 (mGLP-1), as disclosed above.

Suitable to the pharmaceutical composition of this invention, the bioactive polypeptide can comprise FGF21 or a modified FGF21 (mFGF21), as disclosed above.

Suitable to the pharmaceutical composition of this invention, the bioactive polypeptide can comprise a GM-CSF or a modified GM-CSF (mG-CSF), as disclosed above.

Suitable to the pharmaceutical composition of this invention, the bioactive polypeptide can comprise sdAb or a modified sdAb (msdAb), as disclosed above.

As mentioned above, the fusion protein can further comprise a targeting agent covalently linked to the AAT or mAAT polypeptide, the bioactive polypeptide, or a combination thereof.

This invention is also directed to a protein composition comprising a mAAT polypeptide or a functional variant thereof, wherein the mAAT polypeptide or the functional variant thereof is free from cysteine amino acid residue, the functional variant can have at least 85% sequence identity of the mAAT polypeptide and wherein the mAAT polypeptide and the functional variant each is free from serine protease inhibitor activity. The protein composition can comprise a mAAT having a serine or an alanine mutation at a Z position in the mAAT.

This invention is further directed to a pharmaceutical composition comprising the protein composition disclosed herein.

This invention is further directed to an expression vector comprising a coding region comprising AAT codes encoding an AAT polypeptide or a functional variant thereof, and bioactive polypeptide codes encoding a bioactive polypeptide, wherein the AAT codes and the bioactive polypeptide codes are configured to link together directly or via linker codes encoding a linker peptide having an N-terminal, a C-terminal and 1-50 amino acid residues, and wherein the linker codes are positioned between said AAT codes and the bioactive polypeptide codes.

In examples, the coding region is configured to have the bioactive polypeptide linked to the N-terminal of said linker peptide and the AAT polypeptide linked to the C-terminal of the linker peptide when expressed.

In other examples, the coding region is configured to have the bioactive polypeptide linked to the C-terminal of the linker peptide and the AAT polypeptide linked to the N-terminal of the linker peptide when expressed.

Suitable to the expression vector of this invention, the AAT codes can comprise mAAT codes encoding a mAAT polypeptide or a functional variant thereof, wherein the mAAT polypeptide or the functional variant thereof is free from cysteine amino acid residue, wherein the functional variant has at least 85% sequence identity of the mAAT polypeptide and wherein the mAAT polypeptide and the functional variant each is free from serine protease inhibitor activity.

In the expression vector disclosed herein, the coding region can further comprise bioactive polypeptide codes encoding a bioactive polypeptide, wherein the mAAT codes and the bioactive polypeptide codes are configured to link together directly or via linker codes encoding a linker peptide having 1-50 amino acid residues. The coding region can comprise mAAT codes and the bioactive polypeptide codes that are configured to link together directly in one example or configured to link together via linker codes encoding a linker peptide in another example. The mAAT codes can encode a mAAT polypeptide having a serine or an alanine mutation at a Z position in the mAAT.

In one example, the coding region can be configured to have the bioactive polypeptide linked to the N-terminal of the linker peptide and the mAAT polypeptide linked to the C-terminal of the linker peptide when expressed. In another example, the coding region can be configured to have the bioactive polypeptide linked to the C-terminal of the linker peptide and the mAAT polypeptide linked to the N-terminal of the linker peptide when expressed.

In one embodiment, the bioactive polypeptide codes are configured to encode a bioactive polypeptide comprises a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof. Any codes encoding the aforementioned bioactive polypeptides can be suitable. In examples, the bioactive polypeptide can comprise Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

The bioactive polypeptide can have a molecular weight in a range of from 100 to 500,000 Daltons in one example, 100 to 250,000 Daltons in another example, 100 to 150,000 Daltons in yet another example, 100 to 100,000 Daltons in yet another example, 100 to 75,000 Daltons in yet another example, 100 to 50,000 Daltons in yet another example and 100 to 25,000 in yet a further example. In one particular example, a bioactive polypeptide can have a molecular weight in a range of from 100 to 25,000 Daltons. In yet further examples, the bioactive polypeptide can have a molecular weight in a range of from 100 to 24,000 Daltons. In yet further examples, the bioactive polypeptide can have 0 to 3 disulfide bonds. In yet further examples, the bioactive polypeptide can have a molecular weight in a range of from 100 to 24,000 Daltons, 0 to 3 disulfide bonds or a combination thereof.

In a further embodiment, the bioactive polypeptide comprises an interleukin-2 (IL-2) or a modified IL-2 (mIL-2). The mIL-2 can comprise a serine or an alanine mutation at an X position in the mIL-2. In yet further examples, the mAAT codes can comprise codes encoding a serine or an alanine mutation at a Z position in the mAAT and the bioactive polypeptide codes can comprise codes encoding a serine or an alanine mutation at a X position in the mIL-2.

In yet a further embodiment, the coding region can comprise codes identified in SEQ ID. 7 encoding a fusion protein comprises a mIL-2, a short linker peptide GSTSGS and a mAAT or SEQ ID. 8 encoding a fusion protein comprises a mIL-2, a long linker peptide GGGGSGGGGS and a mAAT.

Suitable to the expression vector of this invention, the bioactive polypeptide can comprise an interleukin-15 (IL-15) or a modified IL-15 (mIL-15).

Suitable to the expression vector of this invention, the bioactive polypeptide can comprise a G-CSF or a modified G-CSF (mG-CSF).

Suitable to the expression vector of this invention, the bioactive polypeptide can comprise IFN-α2 or a modified IFN-α2 (mIFN-α2).

Suitable to the expression vector of this invention, the bioactive polypeptide can comprise IFN-81 or a modified IFN-81 (mIFN-β1).

Suitable to the expression vector of this invention, the bioactive polypeptide can comprise GLP-1 or a modified GLP-1 (mGLP-1).

Suitable to the expression vector of this invention, the bioactive polypeptide can comprise FGF21 or a modified FGF21 (mFGF21).

Suitable to the expression vector of this invention, the bioactive polypeptide can comprise sdAb or a modified sdAb (msdAb).

Suitable to the expression vector of this invention, the coding region can further comprise targeting agent codes encoding a target agent polypeptide linked to the AAT polypeptide, the bioactive polypeptide, or a combination thereof.

The expression vector disclosed herein can be configured to express the coding region in a prokaryotic organism, a eukaryotic organism, a virus system, a cell culture system, a cell-free expression system, bacteria, yeast, insect cells, plant, mammalian cells, or a combination thereof. The expression vector can be configured to express the coding region in a cell-free system in one example, in bacteria E. coli in another example, in yeast in yet another example, in mammalian cells in yet another example, and in a virus-host system in a further example. The expression vector can also be configured to express the coding region a combination of system, such as a vector having both E. coli and mammalian expression cassette including promoters, enhancers, inducing sequence, terminators, poly(A) or other necessary elements for expression that are known to those skilled in the art.

The expression vector can be configured based on a host of choice. Typical hosts can include: bacteria, yeast, insect cells, plant, and mammalian cells. The selection of host or hosts can be made based on a number of factors, such as nature of the protein of interest, desired expression yield, development time, availability of expression vector(s) and other technical and production factors.

Bacteria host as an expression system offer some important advantages including high protein yield, fast development cycle, low production cost, in-depth knowledge of the protein expression regulation, and wide availability of expression vectors. However, bacteria host have certain disadvantages. First, many mammalian proteins expressed in E. coli are in insoluble form (inclusion body) and therefore requiring refolding process to obtain a soluble protein. Currently, there is no universal procedure for protein refolding and requires empirical developments to establish a high yield refolding procedure. Second, as a prokaryotic organism, proteins expressed in bacteria are located in the cytosol, which is a reductive environment preventing protein disulfide bonds formation that is required for correct protein folding. Since most eukaryotic secreted proteins contain disulfide bonds, eukaryotic proteins expressed in bacteria often require additional step to form disulfide bonds that is required for their functions. This can be a challenge especially when a protein contains multiple disulfide bonds. Third, a protein expressed in bacteria typically does not contain correct post translational modification, such as glycosylation or phosphorylation, which may affect its biological activity. In bacteria expression system, E. coli is the most widely used, although Bacillus subtilis and other bacteria can also be used.

Three types of yeasts are commonly used as host cells for protein production: Pichia pastoris, Saccharomyces cerevisiae and Kluyveromyces lactis. Although yeast expression systems have the benefit of high biomass, easy genetic manipulation, and the possibility to express secreted proteins, some drawbacks of the yeast expression system limit its wider use. For example, yeast N- and O-glycosylations are different from that in mammalian cells. That may lead to proteins with yeast glycosylations immunogenic in other organism, such as humans. In addition, expression levels of many mammalian proteins in yeast are relatively low compared to that in other hosts.

Insect cells can also be used for protein expression. The commonly used insect cell lines include such as Spodoptera frugiperda, derived from Lepidopterans (moths and butterflies) and Baculovirus, a rod-shaped virus that can infect insect cells. The virus derived shuttle vector is called bacmid. Insect cells can grow fast without the expensive serum normally needed to boost cell growth. The proteins are often expressed in a soluble form with glycosylation, although the pattern of the glycan may be different from that expressed in mammalian cells.

Similarly, many types of plants can be used for the protein expression and many plant expression vectors are available.

Mammalian cells are used for the production of most therapeutic protein products. Although Hela cell, HEK293 cells, COS cells and many other mammalian cells have been developed for protein production, the Chinese hamster ovary (CHO) cells have become a de facto standard host for the biopharmaceutical industry for the production of therapeutic proteins. CHO cells can be adapted to a serum-free media and grow in suspension to a high cell density (>2×10⁷). Yield of protein can reach as high as 10 g/L for antibodies. The protein products are often expressed in correctly folded soluble forms with the glycosylation similar to its native forms for proteins originated from mammalians. The disadvantage of the CHO expression system can include long development cycle time, high cost of cell culture media and complexity of manipulation and operation that typically require high level of technical skills.

The cell-free system has been used for protein productions at a small scale. With high reagent costs and relatively low yield, the use of the cell-free system is often very limited.

The expression vector, such as plasmid or a virus-based expression vector, often contains an E. coli replication origin (PUC Ori) and an E. coli selection marker (AMP and KAN are most often used) to facilitate the cloning process that is carried out in E. coli. It can also contain a selection marker for the selected host if the expression host other than E. coli. For example, antibiotic neomycin resistant marker NeoR can be used for many different host cells, DHFR and GS are used in CHO expression vector. A replication origin sequence for the host cells is also needed in the expression vector if it is not integrated into host genome.

An expression vector can comprise an expression cassette that comprises a promoter, an enhancer, and a translation initiation site (Kozak sequence for mammalian cell and The Shine-Dalgarno sequence for E. coli). These elements can often be located before the first codon ATG, although an enhancer in the mammalian system may be located in the middle or after the coding region. There are often suitable restriction sites for the insertion of the cDNA sequence coding for a protein of interest. The cDNA coding sequence can be obtained by chemical gene synthesis or by a PCR amplification from a gene template. The expression cassette can further comprise a polyadenylation site to ensure the proper processing of mRNA at the end of the coding sequence after a stop codon, such as TAA. The expression cassette can further comprise a signal sequence that is included in the coding region if the protein of interest is aimed to secrete out of host cells into media.

The promoter can include a strong promoter, such as T7 promotor for E. coli, AOX1 promoter for Pichia pastoris; pPolh promoter for Baculovirus and CMV promoter for CHO cells. Many other promoters can be used to achieve a different level of expression.

An expression vector can be configured to express constitutively or inducibly depending on the selection of promoter. A constitutive expression under a strong promoter can lead to the accumulation of a large amount protein products during the course of cell growth. For example, the expression of recombinant antibodies under CMV promoter in CHO cells is constitutive. The antibody products are secreted into culture media continuously. For protein expression in E. coli or yeast, an inducible promoter can be used. In one example, proteins expression in E. coli can be under the control of both lac operon and a T7 promoter. The gene expression can be turned on after the addition of an inducer, such as IPTG (isopropyl-β-D-thiogalactoside), into growth media. In another example, protein expression in yeast Pichia pastoris can be under the control of an AOX1 promoter that can be induced by the addition of methanol in growth media.

In addition to elements in expression cassette such as a promoter and an enhancer, the expression of a recombinant protein can also be affected by the coding sequence. Changing the cDNA sequence by codon optimization can sometimes increase the protein yield by many folds. The increase of the expression yield is often due to the elimination of rarely used codon in the host cell and elimination of certain mRNA structure that may have an inhibitory effect on translation. The expression vector can comprise a coding region having optimized codons for producing a fusion protein of this invention in E. coli host. In one example, the expression vector can comprise a coding region having optimized codons encoding the AAT or mAAT polypeptide. In another example, the expression vector can comprise a coding region having optimized codons encoding the bioactive polypeptide. In yet another example, the expression vector can comprise a coding region having optimized codons encoding the mIL-2 polypeptide. In a further example, the expression vector can comprise a coding region having optimized codons encoding the AAT or mAAT and the mIL-2 polypeptide.

This invention is further directed to a process for producing a fusion protein, the process comprising:

expressing an expression vector comprising a coding region encoding the fusion protein in a host to produce a pre-fusion protein;

harvesting the pre-fusion protein from cells of the host, cell lysate of said host, an inclusion body of the host, media culturing the host, or a combination thereof; and

producing the fusion protein from the pre-fusion protein.

Any of the expression vectors disclosed herein can be Suitable to the process. The expression vector can be configured to comprise a coding region encoding a fusion protein comprises an AAT or a mAAT polypeptide and a bioactive polypeptide comprises a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof.

Suitable to the process, the expression vector can comprise bioactive polypeptide comprises Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

Suitable to the process, the bioactive polypeptide can have a molecular weight in a range of from 100 to 500,000 Daltons in one example, 100 to 250,000 Daltons in another example, 100 to 150,000 Daltons in yet another example, 100 to 100,000 Daltons in yet another example, 100 to 75,000 Daltons in yet another example, 100 to 50,000 Daltons in yet another example and 100 to 25,000 in yet a further example. In one particular example, a bioactive polypeptide can have a molecular weight in a range of from 100 to 25,000 Daltons. The bioactive polypeptide can have a molecular weight in a range of from 100 to 500,000 Daltons including each and every aforementioned molecular range. In yet further examples, the bioactive polypeptide can have a molecular weight in a range of from 100 to 24,000 Daltons. In yet further examples, the bioactive polypeptide can have 0 to 3 disulfide bonds. In yet further examples, the bioactive polypeptide can have a molecular weight in a range of from 100 to 24,000 Daltons, 0 to 3 disulfide bonds or a combination thereof.

Suitable to the process of this invention, the bioactive polypeptide comprises an interleukin-2 (IL-2) or a modified IL-2 (mIL-2). The mIL-2 can comprise a serine or an alanine mutation at an X position in the mIL-2. In one example, an expression vector can comprise a coding region comprising mAAT codes encoding a mAAT polypeptide or a functional variant thereof, wherein the mAAT polypeptide or the functional variant thereof is free from cysteine amino acid residue, wherein the functional variant has at least 85% sequence identity of the mAAT polypeptide and wherein the mAAT polypeptide and the functional variant each is free from serine protease inhibitor activity. In another example, the coding region of the expression vector above can further comprise bioactive polypeptide codes encoding a bioactive polypeptide, wherein the mAAT codes and the bioactive polypeptide codes are configured to link together directly or via linker codes encoding a linker peptide having 1-50 amino acid residues. The coding region can comprise mAAT codes and the bioactive polypeptide codes that are configured to link together directly or configured to link together via linker codes encoding a linker peptide. In yet another example, the coding region can be configured to have the bioactive polypeptide linked to the N-terminal of the linker peptide and the mAAT polypeptide linked to the C-terminal of the linker peptide when expressed. In yet another example, the coding region can be configured to have the bioactive polypeptide linked to the C-terminal of the linker peptide and the mAAT polypeptide linked to the N-terminal of the linker peptide when expressed. In a yet another example, an expression vector can comprise mAAT codes encoding a serine or an alanine mutation at a Z position in the mAAT and the bioactive polypeptide codes encoding a serine or an alanine mutation at a X position in the mIL-2 polypeptide. In yet a further example, an expression vector can comprise a coding region comprising codes identified in SEQ ID. 7 encoding a fusion protein comprises a mIL-2 polypeptide, a short linker peptide GSTSGS and a mAAT or SEQ ID. 8 encoding a fusion protein comprises mIL-2, a long linker peptide GGGGSGGGGS and a mAAT polypeptide.

For the process disclosed herein, the coding region encoding the aforementioned fusion protein can comprise a mAAT polypeptide and an interleukin-2 (IL-2) polypeptide or the mAAT polypeptide and a modified interleukin-2 (mIL-2) polypeptide in one example. The coding region encoding the fusion protein can comprise codes identified by SEQ ID. 7 or SEQ ID. 8.

Suitable to the process of this invention, the bioactive polypeptide can comprise an interleukin-15 (IL-15) or a modified IL-15 (mIL-15).

Suitable to the process of this invention, the bioactive polypeptide can comprise a G-CSF or a modified G-CSF (mG-CSF).

Suitable to the process of this invention, the bioactive polypeptide can comprise IFN-α2 or a modified IFN-α2 (mIFN-α2).

Suitable to the process of this invention, the bioactive polypeptide can comprise IFN-β1 or a modified IFN-β1 (mIFN-β1).

Suitable to the process of this invention, the bioactive polypeptide can comprise GLP-1 or a modified GLP-1 (mGLP-1).

Suitable to the process of this invention, the bioactive polypeptide can comprise FGF21 or a modified FGF21 (mFGF21).

Suitable to the process of this invention, the bioactive polypeptide can comprise sdAb or a modified sdAb (msdAb).

Suitable to the process of this invention, the fusion protein can further comprise a targeting agent covalently linked to the AAT or mAAT polypeptide, the bioactive polypeptide, or a combination thereof.

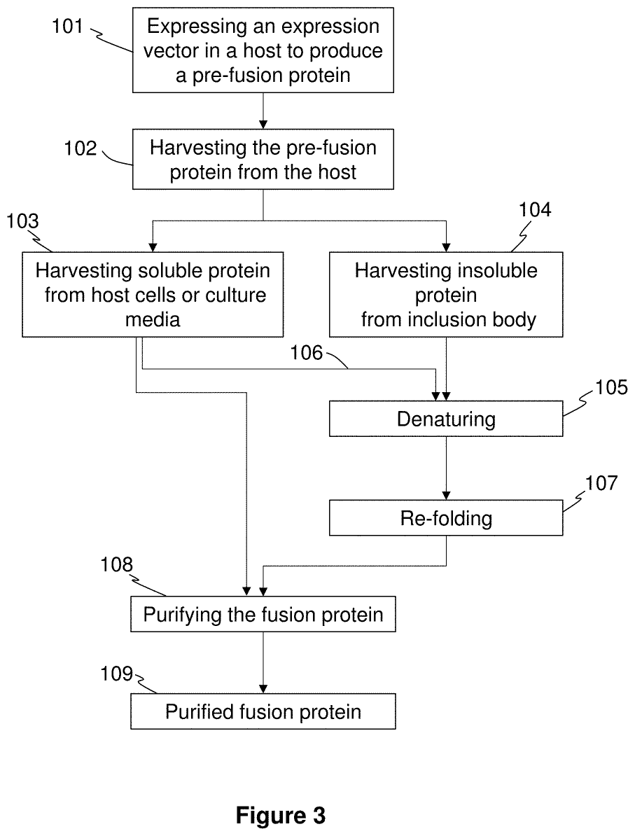

For the process disclosed herein, the host can comprise E. coli cells. An expression vector disclosed herein can be used to express a fusion protein. Based on the expression vector, an induction can be done, such as by adding IPTG (isopropyl-β-D-thiogalactoside) to induce the expression to produce a pre-fusion protein (101) (FIG. 3).

In the process disclosed herein, the pre-fusion protein can be harvested from the inclusion body (102). If the host produces the fusion protein in a soluble form, the fusion protein can also be harvested from cells or culture media (103). If the fusion protein is insoluble and mostly located in inclusion body, cells can be broken and the inclusion body can be harvested (104).

The fusion protein can be produced from the pre-fusion protein by a re-folding process that comprises:

(1) contacting the pre-fusion protein with a denaturing agent;

(2) re-folding the pre-fusion protein by gradually removing the denaturing agent to form the fusion protein; and

(3) purifying the fusion protein.

The inclusion body containing pre-fusion protein can be washed with a wash buffer before being contacted with the denaturing agent. A wash buffer may contain salt, detergent or a combination thereof. In the denaturing step (105), the denaturing agent can comprise a denaturant such as guanidine, guanidine-HCl, urea or a combination thereof, and a reducing agent such as dithiothreitol (DTT), mercaptoethanol or a combination thereof. The denaturing agent can also comprise one or more salts, one or more detergents such as Triton X-100, sodium deoxycholate, or a combination thereof. The denaturing agent can be gradually removed, for example, by dialysis. Once the denaturing agent is removed, the solubilized fusion protein can be refolded (107). The refolded solubilized protein can then be purified (108) to produce a purified fusion protein (109). If desired, soluble fusion can be optionally denatured and refolded (106) to modify or improve protein structures. The soluble protein can also be purified (108) directly without re-folding.

The fusion proteins can be purified using ion exchange chromatography, such as a strong anion exchange or a weak anion exchange chromatography. HiTrap Q HP anion exchange chromatography column (available from GE Health Life Sciences, Pittsburgh, Pa., USA) can be suitable as a strong anion exchange chromatography. HiTrap DEAE Sepharose FF (also available from GE Health Life Sciences) can be an example suitable for a weak anion exchange chromatography. The proteins can be loaded on a Q column or a DEAE column and eluted out according to manufacturers' instructions.

This invention is further directed to a method for treating a disease in a subject in need thereof. The method can comprise administering the pharmaceutical composition disclosed herein to the subject.

Any of aforementioned pharmaceutical compositions of this invention can be suitable. The pharmaceutical composition can comprise a fusion protein comprises an AAT or mAAT polypeptide and a bioactive polypeptide. The pharmaceutical composition can comprise a fusion protein comprises an AAT or mAAT polypeptide, a bioactive polypeptide and a linker positioned between the AAT or mAAT polypeptide and the bioactive polypeptide, as disclosed above. The pharmaceutical composition can comprise a fusion protein comprises an AAT or mAAT polypeptide with a serine or an alanine residue at the Z position, an IL-2 polypeptide with a serine or an alanine residue at the X position and a linker peptide.

In another example, a fusion protein can comprise a mIL-2 polypeptide with X=S or X=A, a short linker and a mAAT polypeptide with Z=S or Z=A (such as SEQ ID. 5). In yet another example, a fusion protein can comprise a mIL-2 polypeptide with X=S or X=A, a long linker and a mAAT polypeptide with Z=S or Z=A (for example, SEQ ID. 6).

Suitable to the method of this invention, the fusion protein can comprise an AAT or a mAAT and a bioactive polypeptide comprising Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-61), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

Suitable to the method, the bioactive polypeptide can have a molecular weight in a range of from 100 to 500,000 Daltons in one example, 100 to 250,000 Daltons in another example, 100 to 150,000 Daltons in yet another example, 100 to 100,000 Daltons in yet another example, 100 to 75,000 Daltons in yet another example, 100 to 50,000 Daltons in yet another example and 100 to 25,000 in yet a further example. In one particular example, a bioactive polypeptide can have a molecular weight in a range of from 100 to 25,000 Daltons. In yet further examples, the bioactive polypeptide can have a molecular weight in a range of from 100 to 24,000 Daltons. In yet further examples, the bioactive polypeptide can have 0 to 3 disulfide bonds. In yet further examples, the bioactive polypeptide can have a molecular weight in a range of from 100 to 24,000 Daltons, 0 to 3 disulfide bonds or a combination thereof.

In the method disclosed herein, the pharmaceutical composition can be administered to the subject via intravenous (IV) injection, subcutaneous (SC) injection, intramuscular (IM) injection, intradermal (ID) injection, or a combination thereof. The pharmaceutical composition can be administered to the subject via intravenous (IV) injection in one example, subcutaneous (SC) injection in another example, intramuscular (IM) injection in yet another example, intradermal (ID) injection in yet another example, or a combination thereof in a further example.

In the method disclosed herein, the pharmaceutical composition can be administered to the subject via a local injection to deliver the pharmaceutical composition into or adjacent to a target or a disease location, such as a tissue, a lesion, an infection site or a tumor. The pharmaceutical composition can also be encapsulated or conjugated with nano-materials such as polymer nanoparticles, liposomes or a combination thereof. The pharmaceutical composition can also be administered locally via implantation of a device containing the pharmaceutical composition intra- or adjacent to the disease location.

For the method disclosed herein, the disease can be a cancer, an autoimmune disease, diabetes, vasculitis, heart disease, virus infection, or a combination thereof. In one example, the pharmaceutical composition comprises a mAAT-antibody fusion protein for cancer immunotherapy. The antibody can be a mAb or a polyclonal antibody, suitable for cancer immunotherapy, such as a PD-1 antibody, a PD-L1 antibody, a checkpoint inhibitor antibody, or a fragment of each antibody thereof.

One advantage of the fusion protein of this invention is to enhance the activity, stability, bioavailability or a combination thereof, of the bioactive polypeptide.

This invention can be used as a new fusion protein platform for producing fusion proteins of fully human origin. The fusion proteins can be expressed and produced in a microorganism, such as E. coli, which has the advantage of short developing time, low manufacturing cost and a high production yield. Although several well-known fusion protein platforms of human proteins are available, such as human serum albumin (HSA), immunoglobulin Fc fragment or transferrin, they are generally not well expressed in E. coli. On the other hand, the commonly used fusion protein platforms in E. coli, such as GST, MBP, are not human proteins, which can have immunogenicity if the fusion protein is used for therapeutic purposes in human patients. Therefore, there is a need for a new fusion protein platform that is of human origin and can be expressed with a good yield in E. coli or similar microorganism. The fusion protein platform of this invention can provide advantages over these existing platforms.

The AAT protein, similar to some other members of serpin proteins, has a very unique property of having a “flexible” conformation. The AAT protein can change its conformation from S (stressed) to R (relaxed) spontaneously or upon interaction with other proteins. The IL-2 protein comprises a four-alpha helix bundle which are viewed as a “rigid” structure. Not wishing to be bound by a particular theory or mechanism, applicants believe that the fusion protein of this invention comprising mAAT and mIL-2 polypeptides can provide a novel ligand for the IL-2 receptor (IL-2R) that contains a rigid “head” and a flexible “body”. Such novel ligand can provide some special properties or functions in the IL-2R binding, T-cell activation and other biological and physiological activities, some of which are exemplified hereafter.

In addition to the aforementioned bioactivity, Applicants also unexpectedly discovered that the mutations replacing the Cys residue at the X and the Z positions, such as those mutations with X=S or A and Z=S or A, significantly increase soluble protein yields after denaturing and refolding the fusion proteins expressed from E. coli host system.

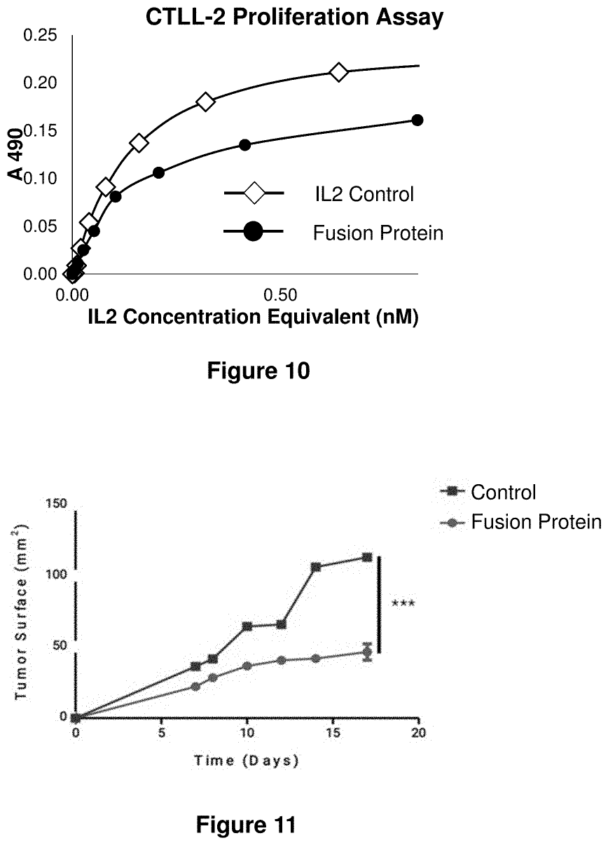

Applicants further unexpectedly discovered that mAAT-mIL-2 fusion proteins of this invention provide activities in T-cell stimulation comparable to native IL-2 (FIG. 10). This is unexpected since it is known that polyethylene glycol (PEG)-conjugated (PEGylated) interleukin 2 molecules (PEG-IL2) have activities about 10 to 100-fold less than the native IL-2 based on the EC₅₀ values (Charych, et a., Clin Cancer Res. 2016 Feb. 1; 22(3):680-90). Applicants unexpectedly discovered that the mAAT-mIL-2 fusion protein of this invention further showed significant tumor inhibition activity in a mouse tumor model, as exemplified hereafter (FIG. 11). Interleukin 2 is a well-known cytokine that plays a key role in regulating the immune system. In vivo, the activity of IL-2 molecule is short-lived, which limits its use in therapeutic applications in treating disease such as cancer or auto-immune disease. Further, the use of IL-2 molecule as a therapeutic agent has some serious side effects such as capillary leak syndrome. Not wishing to be bound by any particular theory or mechanism, Applicants believe that the fusion protein of this invention provides a better in vivo stability and protein conformation resulting in excellent T-cell activation activity and extended duration of the action. Such feature of the fusion protein of this invention can result in improved pharmaceutical effects for treating a disease, such as a cancer and autoimmune disease. Further clinical studies and developments are still needed.

A further advantage is that the fusion protein of this invention using the recombinant technology can be highly reproducible, unlike other protein modification technologies, such as conjugated proteins, for example, PEGylated proteins or HSA encapsulated proteins, that can have many variations from batch to batch. Once an optimized fusion protein peptide sequence is selected, the exact protein can be produced reproducibly using the fusion protein platform of this invention.

As exemplified in representative examples below, a large number of AAT (or mAAT) fusion protein expression constructs were produced, expressed in E. coli cells to produce pre-fusion protein, refolded and purified. The fusion proteins were produced with: (1) different classes of bioactive polypeptides, such as cytokines (IL2, IL15), interferons (IFN-alpha-2, IFN-beta-1), growth factors (G-CSF, GM-CSF, FGF21), hormones (GLP-1) and single-domain antibody (ALX-81); (2) both N- and C-terminal fusions; (3) different linkers; (4) various mutations in the AAT sequence at the Z position, such as Z=Ser, Cys, Ala, etc.; and (5) various mutations in the bioactive polypeptide sequences at the X position, such as X=Ser, Cys, Ala for IL2, X=Asp, Asn for ID 5, and others. The fusion proteins were expressed and produced in host cells, refolded and purified. The purified fusion proteins were then used for functional assays. These representative examples demonstrate yet a further advantage of this invention that various bioactive polypeptides in different classes can each be fused with an AAT or mAAT polypeptide to generate a fusion protein that can have increased molecular weight while retain the biological activity of the bioactive polypeptide. The fusion proteins of this invention can provide enhanced in vivo stability and protein conformation for improved function.

Although representative examples of the fusion proteins are exemplified in this disclosure, it is understood that further fusion proteins with additional or variants of combinations or modification can be made without departing from the spirits of this invention. The combination or modifications can include, but not limited to, different classes of bioactive polypeptide or agent, different linkers or various linker sizes, different formats of fusions (N- or C-terminal), mutation or modification variants of AAT polypeptides and mutation and modification variants of bioactive polypeptides or bioactive agents.

### Examples

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### 1. Methylation and Mutagenesis Reactions

Preparing 25×SAM: Solution was prepared by dilution from a 200×SAM (S-adenosine methionine) solution of the GENEART® Site-Directed Mutagenesis PLUS Kit available from Invitrogen Lifetechnologies™ (Carlsbad, Calif., USA, under respective trademark or registered trademark), in distilled sterile water within a few hours prior to each mutagenesis procedure.

DNA Polymerase: The DNA polymerase used was the AccuPrime™ Pfx DNA Polymerase available from ThemoFisher (Carlsbad, Calif., USA, under respective trademark) for high fidelity, high-specificity amplification of DNA fragments.

Amount of Plasmid: About 20-50 ng or less plasmid DNA per 50 μL of methylation reaction/PCR amplification was used.

Mutagenesis reactions were conducted according to the Kit manufacturer's instruction.

Recombination Reaction: The in vitro recombination reaction was done for multi-site and single-site mutagenesis reactions using corresponding PCR products. For single-site mutagenesis, the recombination reaction was observed to boost mutagenesis efficiency and increase the colony yield 3 to 10-fold. Following procedure was used for the recombination reaction:
1) Components below were added in a tube for each 10-μL recombination reaction using multiple PCR products:
   PCR water 34 μL (adjust the volume of PCR water based on the volume of PCR products used below to reach a total volume of 5 μL before the Enzyme Mix)
   PCR product 2 μL
   GENEART® 2× Enzyme Mix 5 μL (final concentration: 1×)
2) Mixing well and incubate at room temperature for 15 minutes;
3) Stopping the reaction by adding 1 μL 0.5 M EDTA. Mixing well and place the tubes on ice; and
4) Placing the tubes on ice and immediately proceed to transformation.

Transformation with Mutagenesis Reaction Products:

1) Using 50 μL of One Shot® MAX Mfficiency® DH5α™-T1® competent cells, available from ThemoFisher Scientific, Carlsbad, Calif., USA, under respective trademarks and registered trademarks, for transformation;

2) Transferring about 3 μL of the recombination reaction prepared above directly into the competent cells to transform the cells according manufacturer's instruction.

3) Removing the vials from ice and adding 250 μL of pre-warmed S.O.C. medium to each vial and incubating at 37° C. for exactly 1 hour in a shaking incubator set to 225 rpm. Plate 30-100 μL of the cell suspension on LB agar plates containing the appropriate antibiotics.

4) Storing the remaining transformation reaction at 4° C. and incubating the plates at 37° C. for 16-18 hours.

5) Selecting 3 to 5 colonies and analyzing by plasmid isolation, PCR, and sequencing.

2. Construction of mIL2-Linker1-mAAT (Short Linker) (X=Ser; Z=Ser)

This is an A-ShortLinker-B structure. One cDNA coding for the fusion protein (SEQ ID. 5) with a short linker and X=Ser (actual position 126) and Z=Ser (actual position 372) was experimentally selected based on optimal expression in E. coli. The cDNA sequence is different from the original human cDNA for both IL-2 and AAT genes. The signal sequence of the AAT was removed. Two unique restriction sites at 5′ and 3′ ends were also included in the synthesized cDNA. The synthesized cDNA was subcloned into a protein expression vector PT88 developed by the Applicants that is similar to PET-28a (Novagen, now part of Merck KGaA, Germany). The PT88 vector contains a T7 promoter under control of lac operon, kanamycin resistant (KanR) selection marker, a PUC replication origin, and restriction sites that matched the restriction sites in cloning (Plasmid 1). The cDNA sequence of the fusion protein coding region in Plasmid 1 is shown as SEQ ID. 7 starting from ATG to TAA corresponding to the start codon and the stop codon, respectively.

3. Construction of mIL2-Linker2-mAAT (Long Linker) (X=Ser; Z=Ser)

This is a cDNA coding for an A-LongLinker-B structure fusion protein (SEQ ID. 6) with X=Ser (actual position 126) and Z=Ser (actual position 376) mutations. It was constructed by removing a 449 bp (base pair) fragment from the Plasmid 1 above by cutting with restriction enzymes SphI and SspI at the position 118-567. A synthesized 461 bp DNA fragment that contains DNA coding for a long linker (GGGGSGGGGS) was inserted to replace the removed fragment to produce a Plasmid 2. The cDNA sequence of the fusion protein coding region in Plasmid 2 is shown as SEQ ID. 8.

### 4. Construction of Other IL2-AAT Fusion Proteins (X=Cys, Ala; Z=Cys, Ala) by Mutagenesis

Additional mutations in the IL-2 and the AAT coding regions were produced by mutagenesis using the Plasmid 1 or 2 as a template and GENEART Site-Directed Mutagenesis Plus Kit (Life Technologies) and AccuPrime Pfx DNA polymerase (Life Technologies) as described above. The mutated plasmids were then transformed into E. coli competent cells Dh5α as described above. Mutations were constructed using following primers. A list of the fusion proteins is shown in Table 2.

### Paired Primers for Producing Desired Mutations

Primer-1, for generating X = Cys
(Fusions 5 and 6):
F
SEQ ID. 17
GTTGGATTACCTTCTgTCAGTCTATCATTTC
39% GC, Tm 55° C.
R
SEQ ID. 18
GAAATGATAGACTGAcAGAAGGTAATCCAAC
39% GC, Tm 53° C.
Primer-2, for generating Z = Cys
(Fusions 7 and 8):
F
SEQ ID. 19
TCAACATCCAACACTgCAAGAAACTGTCGTC
45% GC, Tm 61° C.
R
SEQ ID. 20
GACGACAGTTTCTTGcAGTGTTGGATGTTGA
45%GC, Tm 59° C.
Primer-3, for generating X = Ala
(Fusions 9, 10, 13 and 14):
F
SEQ ID. 21
CGTTGGATTACCTTCgCTCAGTCTATCATTT
42% GC, Tm 58° C.
R
SEQ ID. 22
AAATGATAGACTGAGcGAAGGTAATCCAACG
42% GC, Tm 56° C.
Primer-4, for generating Z = Ala
(Fusions 11-14):
F
SEQ ID. 23
TTCAACATCCAACACgCCAAGAAACTGTCGTC
47% GC, Tm 61° C.
R
SEQ ID. 24
GACGACAGTTTCTTGGcGTGTTGGATGTTGAA
47% GC, Tm 59° C.

For Fusions 13 and 14, two rounds of mutagenesis were conducted: First use Primer 3 pair to make X=Ala mutations using the Fusions 3 and 4 to produce intermediate plasmids Fusions having X=A and Z=Ser. Then, the intermediate plasmids were used as templates with Primer-4 to produce the Fusions having the double mutations X=A and Z=A.

TABLE 2
Mutated Fusion Proteins (only the amino
acid residues flanking the X
or the Z positions are shown).

| | | | Z Position |
|---|---|---|---|
| | X Position | | mAAT (256) |
| Fusion Protein | RWITFXQ | | NIQHZKK |
| IL-2 (125) | SIISTLT | Linker | LSSWVL |
| Fusion 1 | SEQ ID. 9 | GSTSGS | SEQ ID. 12 |
| (Comparative 1) | X = C | | Z = C |
| Fusion 2 | SEQ ID. 9 | GGGGSG | SEQ ID. 12 |
| (Comparative 2) | X = C | GGGS | Z = C |
| Fusion 3 | SEQ ID. 10 | GSTSGS | SEQ ID. 13 |
| (Plasmid 1) | X = S | | Z = S |
| SEQ ID. 7 | | | |
| Fusion 4 | SEQ ID. 10 | GGGGSG | SEQ ID. 13 |
| (Plasmid 2) | X = S | GGGS | Z = S |
| SEQ ID. 8 | | | |
| Fusion 5 | SEQ ID. 9 | GSTSGS | SEQ ID. 13 |
| (Comparative 3) | X = C | | Z = S |
| Fusion 6 | SEQ ID. 9 | GGGGSG | SEQ ID. 13 |
| (Comparative 4) | X = C | GGGS | Z = S |
| Fusion 7 | SEQ ID. 10 | GSTSGS | SEQ ID. 12 |
| (Comparative 5) | X = S | | Z = C |
| Fusion 8 | SEQ ID. 10 | GGGGSG | SEQ ID. 12 |
| (Comparative 6) | X = S | GGGS | Z = C |
| Fusion 9 | SEQ ID. 11 | GSTSGS | SEQ ID. 13 |
| | X = A | | Z = S |
| Fusion 10 | SEQ ID. 11 | GGGGSG | SEQ ID. 13 |
| | X = A | GGGS | Z = S |
| Fusion 11 | SEQ ID. 10 | GSTSGS | SEQ ID. 14 |
| | X = S | | Z = A |
| Fusion 12 | SEQ ID. 10 | GGGGSG | SEQ ID. 14 |
| | X = S | GGGS | Z = A |
| Fusion 13 | SEQ ID. 11 | GSTSGS | SEQ ID. 14 |
| | X = A | | Z = A |
| Fusion 14 | SEQ ID. 11 | GGGGSG | SEQ ID. 14 |
| | X = A | GGGS | Z = A |

5. Construction of mAAT-Linker-mIL-2 Fusion Proteins

Fusion protein of B-Linker-A structures were constructed by rearranging the AAT and IL-2 polypeptides (Table 3). Fusion 15 has the polypeptide mAAT-Linker-mIL-2 structure with the short linker. Fusion 16 has the polypeptide mAAT-Linker-mIL-2 structure with the long linker.

TABLE 3
Fusion Proteins with B-Linker-A
Structure (only the amino acid
residues flanking the X or the Z
positions are shown).

| | Z Position | | X Position |
|---|---|---|---|
| | mAAT (256) | | IL-2 (125) |
| Fusion | NIQHZKK | | RWITFXQ |
| Protein | LSSWVL | Linker | SIISTLT |
| Fusion 15 | SEQ ID. 13 | GSTSGS | SEQ ID. 10 |
| | Z = S | | X = S |
| Fusion 16 | SEQ ID. 13 | GGGGSG | SEQ ID. 10 |
| | Z = S | GGGS | X = S |

### 6. Polypeptide Sequence Confirmation

Sequences of the proteins were confirmed by LC-MS based peptide mapping. For each of the proteins, about 20 ug of purified protein was denatured by adding guanidine HCl (GuHCl) to 6M. The disulfide bonds in the protein was reduced by the reaction with 1,4-dithiothreitol (DTT, use 20 mM in the reaction at pH 8). The free cysteine was alkylated with iodoacetamide (IOM, 25 mM in the reaction). It is preferred to add IOM with a higher concentration than DTT, since remaining DTT in the reaction mixture need to be titrated by IOM before Cysteine alkylation reaction can happen. The reduction reaction was carried out at 37° C. for 1 hour, and the alkylation reaction was carried out at room temperature for one hour in dark. After alkylation reaction, the sample was dialyzed to remove all salts. Then, the clean protein sample was digested by trypsin (Sequencing Grade Modified Trypsin, Catalog number V511C, Promega, Madison, Wis., USA). The digestion reaction was carried out at 37° C. for 2 hours in 50 mM NH₄HCO₃ at pH 8. The digested sample was then loaded onto a NanoLC-MS system (Agilent HPLC 1100 coupled with Thermo LTQ XL linear Ion Trap Mass Spectrometer) for peptide sequencing. The particular mutation was confirmed based on MS/MS data from the peptide containing the mutated amino acid.

### 7. Expression of the IL2-AAT Fusion Proteins

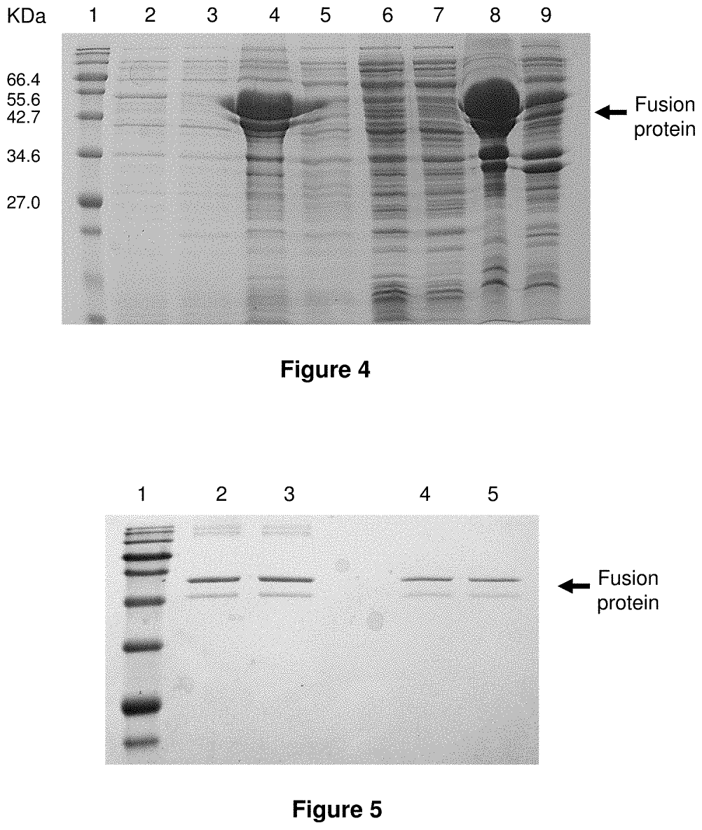

The fusion proteins were under the control of a T7 promotor in the expression vectors. Plasmids encoding the fusion proteins were expressed in E. coli BL21 (available from ThemoFisher) and Origami™ (available from MilliporeSigma, Burlington, Mass., USA, under respective trademark) strains. The transformed E. coli cells were grown in LB+Kanamycin media until the OD of the culture was between 0.8-1.0. IPTG (isopropyl-β-D-thiogalactoside) (0.02 mM) was added to the culture to induce the protein expression. The induction was carried out at 20° C. for 12 hours before harvesting the E. coli cells. An aliquant of cells before and after induction, and samples after cell lysis (insoluble and soluble fractions) were used to run SDS-PAGE. A representative SDS-PAGE gel image is shown in FIG. 4 for the Fusion protein 3 having the structure mIL-2-ShortLinker-mAAT (also referred to as IL2-Linker1-AAT(X=Ser, Z=Ser)).

Legend to FIG. 4: Lane 1, Molecular weight (MW) Marker; Lane 2, BL21 cells before induction; Lane 3, Origami™ cells before induction; Lane 4, BL21 cells after induction; Lane 5, Origami cells after induction; Lane 6, Induced BL21 supernatant after cell lysis; Lane 7, Induced Origami supernatant after cell lysis; Lane 8, inclusion body from induced BL21 cells; Lane 9, Inclusion body from induced Origami cells. The fusion protein had a MW of about 50 KDa.

The results indicated that the fusion protein was expressed at a high yield in BL21 cells and a low yield in Origami stains. In both types of E. coli cells, majority of expressed fusion proteins were in an insoluble form and located in inclusion body.

Fusion proteins having Ala residues at the X and/or Z positions had similar protein expression levels compared to fusion proteins having Ser residue at the X and/or Z positions.

Fusion proteins having both Cys residues at the X and Z positions had lower expression levels in E. coli cells compared to fusion proteins having Ser residue at the X and/or Z positions.

### 8. Refolding of the IL2-AAT Fusion Proteins

Since the expressed fusion proteins were mainly in an insoluble form, refolding is a necessary step to produce active forms of proteins. The fusion proteins from the inclusion body were washed 3 times with 0.5% CHAPS detergent and then solubilized in 6 M guanidine, 10 mM beta-mercaptoethanol. Solubilized protein was diluted 20 folds and then dialyzed in 10 mM Tris buffer pH 8 overnight with three changes of buffer to gradually remove the denaturing agent. The solubility of the protein after refolding was checked with SDS-PAGE after precipitation of insoluble protein. The SDS-PAGE results indicate that significant part of insoluble proteins become soluble after this refolding step. A representative gel image is shown in FIG. 5 for fusion proteins having X=S and Z=S (IL2-Linker1-AAT(X=S, Z=S)).

Legend to FIG. 5: Lane 1, MW marker; Lanes 2 and 3, fusion proteins before refolding step; Lanes 4 and 5, fusion proteins after refolding.

Fusion proteins having one or two Cys residues at the X and/or Z position (Comparatives 1-6) showed precipitations and were not well refolded.

The fusion proteins containing Cys residue at X or Z position produced a lower yield when expressed in E. coli BL21 strain. For Fusion 1, 2, 5, 6, 7, 8 in Table 2 (Comparatives 1-6), although their initial expression levels from E. coli BL21 strain were similar to other fusion proteins (Fusion 3, 4, 9, 10, 11, 12, 13, 14), yields of folded proteins after the refolding step were basically undetectable. About greater than 99% of the proteins in those Comparative examples were precipitated in insoluble forms during the refolding step. In contrast, under the same refolding conditions, the fusion proteins without Cys residues (Fusion 3, 4, 9, 10, 11, 12, 13, 14) refolded well with yields of refolded protein greater than 80%, percentage based on the total protein amount used for refolding.

### 9. Purification of the IL2-AAT Fusion Proteins

The fusion proteins are purified by a strong anion exchange such as HiTrap Q HP anion exchange chromatography column (available from GE Health Life Sciences, Pittsburgh, Pa., USA) or a weak anion exchange chromatography such as HiTrap DEAE Sepharose FF (also available from GE Health Life Sciences). For a strong anion exchange, the protein was loaded on a Q column at pH8.0 and eluted with eluted with 0.5 M to 1 M NaCl in MES buffer pH 6.5. For a weak anion exchange chromatography, the protein was loaded onto a DEAE column in Tris buffer pH 8.0. Fraction elution was performed from 0.1 M NaCl up to 1 M NaCl in 10 mM MES buffer pH 6.5. The fusion protein was eluted at 0.2M and 0.3M NaCl.

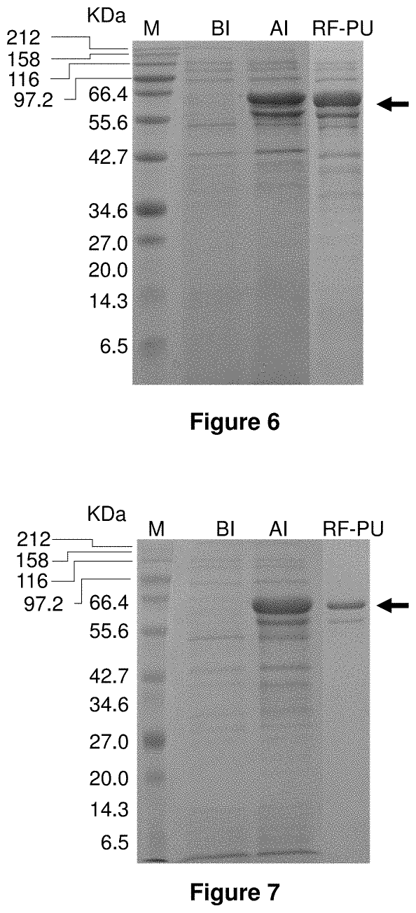

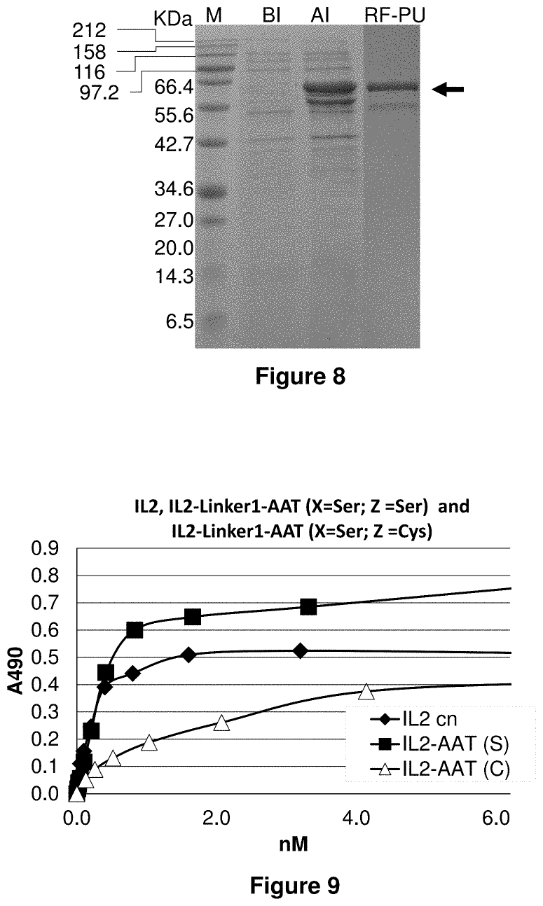

FIG. 6 shows a representative example of expression, refolding and purification of IL2-Linker1-AAT (X=Ser;Z=Cys) fusion protein with Mw of about 60 kDa. FIG. 7 shows a representative example of expression, refolding and purification of IL2-Linker2-AAT (X=Ser;Z=Ser) fusion protein with Mw of about 60 kDa. FIG. 8 shows a representative example of expression, refolding and purification of IL2-Linker2-AAT (X=Ser;Z=Cys) fusion protein with Mw of about 60 kDa.

As used herein throughout this disclosure including all Figures: Molecular weight markers are shown in KDa; BI: Before induction; AI: After induction; RF-PU: Refolding and Purification. The fusion protein is indicated with an arrow.

These data and additional data disclosed above and hereafter demonstrate that various forms of AAT polypeptides can be used to construct fusion proteins with various bioactive polypeptides and the resulted fusion proteins can be expressed, refolded and purified at high yields.

### 10. Characterization of Fusion Proteins

The sequences of the fusion proteins were confirmed by NanoLC-MS-based peptide sequencing. The procedure for the analysis is as following.

Sample Preparation: A fusion protein solution sample was first denatured in 8M urea, with disulfide linkages reduced by DTT and all Cysteine residues alkylated by iodoacetamide. The sample was then cleaned by dialysis to remove all the chemicals and digested with sequencing grade modified trypsin (available from Promega, Madison, Wis., USA) in the digestion buffer (ammonium bicarbonate 100 mM, pH8.5). The peptides from the digestion were completely dried in a SpeedVac device (available from ThermoFisher). The dried sample was then re-dissolved in sample solution (2% acetonitrile 97.5% water, 0.5% formic acid). The re-dissolved protein sample was then analyzed by a NanoLC-ESI-MS/MS system as described before.

NanoLC-ESI-MS/MS Analysis: NanoLC-ESI-MS/MS analysis of digested protein samples was carried out by a high-performance liquid chromatography (HPLC) system (Agilent Technologies, Santa Clara, Calif., USA) with a 75-micrometer inner diameter 8 cm in length reverse phase C18 column. The particle size of the C18 was 3 μM with a pore size of 300 Å. The injection time was about 20 minutes. The HPLC Solvent A was 97.5% water, 2% acetonitrile, 0.5% formic acid. HPLC Solvent B was 9.5% water, 90% acetonitrile, 0.5% formic acid. The gradient time was 60 minutes from 2% Solvent B to 90% solvent B, plus 20 minutes for sample loading and 20 minutes for column washing. The column flow rate was around 800 nanoliter per minute after splitting. Typical injection volume was about 3 ul.

The HPLC system was on-line coupled with an ion trap mass spectrometer (LTQ, ThermoFisher) in a way a sample eluted from HPLC column was directly ionized by an electrospray ionization (ESI) process and enters into the mass spectrometer. The ionization voltage was often optimized each time and normally in a range of 1.2 kv-1.8 kv. The capillary temperature was set at 120° C. The mass spectrometer was set at the data-dependent mode to acquire MS/MS data via a low energy collision-induced dissociation (CID) process. The default collision energy was about 33% and the default charge state was 3. One full scan with 1 micro scan with a mass range of 550 a.m.u to 1800 a.m.u was acquired, followed by one MS/MS scan of the most intense ion with a full mass range and three micro scans. The dynamic exclusion feature was set as following: repeat count of 1 within 0.3 min and exclusion duration of 0.4 min. The exclusion width was 4 Da.

Database Search and Validation: The mass spectrometric data was used to search against the non-redundant protein database (NR database, NCBI) with ProtTech's ProtQuest software suite. After the confirmation of the correctness of the target protein, a small database containing the particular amino acid sequences of fusion proteins were used in the database search to validate the whole fusion protein sequences, including mutations at the X and Z positions and the linker sequences.

Results: The sequences of all fusion proteins were confirmed. Some fusion proteins with truncated N-terminals was observed. For example, some of the first Met residue was truncated in the fusion proteins produced. Such truncations exhibited no effect on the function of the fusion proteins tested. The percentage of truncated protein was different among different fusion proteins.

11. Cell Based Assay of mIL2-Linker-mAAT Fusion Proteins

The activity of the mIL2-Linker-mAAT fusion proteins for the stimulation of T cells was measured using CTLL-2 cell-based colorimetric MTS assay for assessing cell metabolic activity. In the presence of phenazine methosulfate, NAD(P)H-dependent cellular oxidoreductase enzymes may convert MTS (3-(4,5-dimethylthiazol-2-yl)-5-(3-carboxymethoxyphenyl)-2-(4-sulfophenyl)-2H-tetrazolium) into a formazan product, which has an absorbance maximum at 490 nm in phosphate-buffered saline.

CTLL-2 cells were cultured in RPMI 1640 supplemented with 10% fetal bovine serum, and 33 ng/ml IL-2. The cells were harvested in their logarithmic phase and washed two times with an initial volume of Hanks' balanced salt solution (HBSS) with centrifugations at 1000 rpm, 5 min, and incubated for 4 h in RPMI 1640 supplement with 10% FBS (without IL-2) at 37° C., 5% CO₂. The IL-2 control and fusion protein 3 (mIL2-mAAT with a short linker prepared above) were diluted to an initial concentration of 100 ng/ml in the assay medium and followed by serial dilutions and added to the wells in 100 μl of the assay medium. The prepared cell suspension was seeded immediately in the wells of a 96-well plate in 100 μl of the assay medium and incubated at 37° C., 5% CO₂ for 48 h. After the 48 h incubation period, the MTS assay solution was added (20 μl/well) and incubated for another 4 h at 37° C. and 5% CO₂. The plate was then read at 490 nm by a Bio-Rad Model 680 Microplate Reader (available from Bio-Rad, Hercules, Calif., USA) that measures the absorbance of the contents in the wells of a 96-well microtitration plate at 490 nM. Representative examples of activities of IL2 control (IL2 CN, Solid Diamond), IL2-Linker1-AAT (X=Ser;Z=Ser) (IL2-AAT(S), Solid square) and IL2-Linker1-AAT (X-Ser,Z=Cys) (IL2-AAT(C), Open triangle) measured using CTLL2 cell proliferation assay (FIG. 9). Representative results at lower IL2 concentrations are shown in FIG. 10 for the fusion protein Fusion 3 (SEQ ID. 5 having a short linker peptide (Linker1) and IL2 mutation at the X position and AAT mutation at the Z position (X=S and Z=S). The IL-2 control used was purchased from R&D Systems (Catalog Number 202-IL, R&D Systems Inc., Minniapolis, Minn., USA). The data shown that the fusion protein of this invention had T-cell activation activity comparable to the native IL-2 control.

The EC50 of the various mIL2-AAT fusion proteins were measured using the CTLL-2 cell-based assay and listed in Table 4. FIG. 9 and FIG. 10 show the cell proliferation curves of the CTLL-2 assay with recombinant rhIL2 (IL2 CN), mIL2-linker1-AAT (X=Ser, Z=Ser) and mIL2-linker1-AAT(X=Ser, Z=Cys).

TABLE 4
EC50 of Various IL2-AAT Fusion Proteins.

| Fusion Protein | EC50 (nM) |
|---|---|
| IL2(X = Ser)-linker1-AAT (Z = Ser) | 0.14 |
| IL2(X = Ser)-linker2-AAT (Z = Ser) | 0.15 |
| IL2(X = Cys)-linker1-AAT (Z = Ser) | 1.1 |
| IL2 (X = Ser)-linker1-AAT (Z = Cys) | 1.5 |
| IL2(X = Ala)-linker1-AAT(Z = Ser) | 1.95 |
| IL2(X = Ser)-linker1-AAT(Z = Ala) | 3.2 |
| IL2 (X = Ala)-linker1-AAT(Z = Ala) | 1.9 |

A fusion protein with the highest activity in cell-based assay was used for animal study described below.

12. Anti-Tumor Activity of the mIL2-mAAT Fusion Proteins in Mouse Tumor Model

The anti-tumor activity of the IL-2-AAT fusion proteins was examined using a tumor model Foxp3^{YFP-cre} mouse available from The Jackson Laboratory, Bar Harbor, Me., USA. About 1×10⁶ MCA205 sarcoma tumor cells were implanted into Foxp3^{YFP-cre} mice. When tumor size reached 50×50 mm² (14 to 20 days), the mice were received 10 ug mIL2-mAAT fusion protein or the same volume of PBS as a control. Tumor growths were assessed every three days. Mice received another fusion protein or PBS injection during the following week. About 10 mice were used in testing of the fusion protein (5 in the drug group, 5 in control group). To minimize the number of sacrificed mice in the experiments, we only tested the fusion protein formats with high express/refolding yield and good T cell stimulation activity in the animal model study. The result from mIL2-Linker1-mAAT (Fusion 3 prepared above, with a short linker peptide, X=Ser and Z=Ser) is shown in FIG. 11.

### 13. The Protease Inhibition Activity of the IL2-AAT Fusion Proteins

AAT in its native form can inhibit serine protease activity (such as trypsin, elastase, chymotrypsin) by covalently linked to the protease. We tested the protease inhibition of the IL2-linker-AAT fusion protein by incubation with excess amount of the fusion protein with serine proteases: elastase and chymotrypsin, as well as incubation with a protease and a protease substrate, a denatured monoclonal antibody, anti-CD134 antibody, purchased from Biorbyt Ltd with catalog number orb303967 (Biorbyt Ltd., Cambridge, United Kingdom). No activity for serine protease inhibition was detected for IL2-Linker1-AAT (short linker, X=Ser, Z=Ser) (Fusion 3). The presence of the mAAT fusion protein had no inhibitory effect on protease activities of the proteases tested. The results demonstrate that the fusion protein is free from protease inhibitor activity.

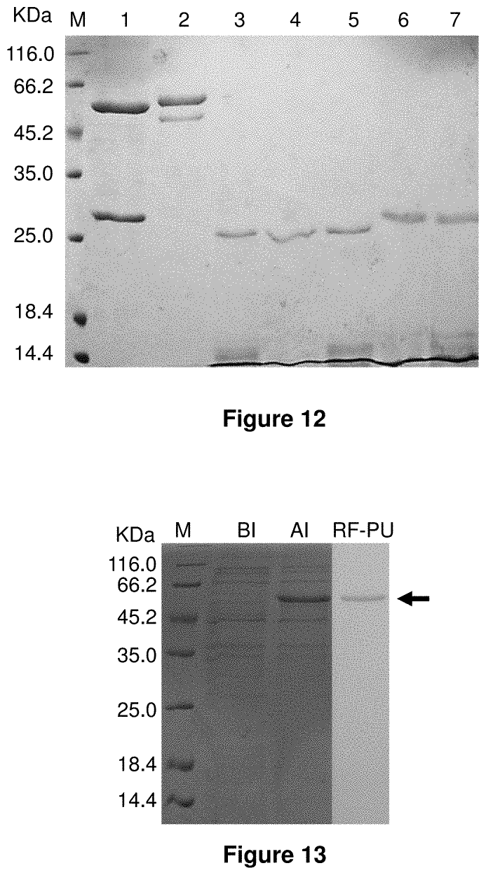

Some representative data are shown in FIG. 12:

Lane 1, control containing antibody sample as protease substrate;

Lane 2, IL2-Linker1-AAT fusion protein identified above;

Lane 3, substrate plus trypsin with the substrate digested by trypsin after incubation shown as the disappearance of the substrate band;

Lane 4, the fusion protein plus trypsin showing the disappearance of the fusion protein band indicating that the fusion protein was digested by trypsin due to the lack of the protease inhibition activity;

Lane 5: the substrate, the AAT fusion protein and trypsin showing the disappearance of the fusion protein and the substrate bands indicating the intact protease activity of trypsin due to the lack of the inhibition;

Lane 6 and Lane 7, similar to lanes 4 and 5 with the trypsin replaced with elastase, another serine protease.

Above results indicate that IL2-linker1-AAT(Z=Cys) fusion protein does not retain protease inhibitor activity. The IL2-linker2-AAT(Z=Ser) was also tested and the results were similar.

14. Construction, Expression, Refolding, Purification and Characterization of mIL15-Linker-mAAT Fusion Proteins
14(a). Construction, Expression and Purification of mIL15-Linker2-mAAT (X=Asp, Z=Ser)

The expression vector for this mIL15-AAT fusion was constructed by replacing a XbaI-DraIII DNA fragment in Plasmid 2 with a synthesized DNA fragment ID 5 cDNA sequence containing the ID 5 cDNA sequence (IL15 amino acid 73 position X=Asp), wherein the X position in IL15 is amino acid position 73. The resulted cDNA sequence of the fusion protein mIL15-linker2-AAT(X=Asp, Z=Ser) is shown in SEQ ID. 25, which was confirmed by DNA sequencing. The sequence of the expressed protein is in SEQ ID. 26, which is confirmed by LC-MS/MS as described above. The expressed fusion protein was mainly located in the cytosol of E. coli cells. The fusion protein was active in CTLL-2 cell based biological activity assay using the procedure described above. The soluble protein may be purified without conducting the protein refolding step. Although it might be beneficial without carrying out protein refolding step, it is challenging to purify the soluble protein from E. coli cytosol to a high purity that is suitable for pharmaceutical usage.

14(b). Construction, Expression and Purification of mIL15-Linker2-mAAT (X=Asn, Z=Ser)

The expression vector for this fusion protein was generated by site-directed mutagenesis of the expression vector from 14(a) (SEQ ID. 25 for cDNA sequence) with the primer pair of SEQ ID 59 and SEQ ID. 60, following the procedure described above. The resulted cDNA sequence of the fusion protein mIL15-linker2-AAT(X=Asn, Z=Ser) is shown as SEQ ID. 27, which was confirmed by DNA sequencing. The sequence of the expressed protein is shown in SEQ ID 28, which was confirmed by LC-MS/MS described above. The expression of this construct in E. coli BL21 cells yielded a high level of the fusion protein after the IPTG induction as described above. Different from the fusion protein described in 14(a), this fusion protein was mainly located in inclusion body. Using the procedure described above, the fusion protein was refolded with a high yield, and further purified with an ion-exchange column Q to a high purity. Representative data on the whole cell lysate before and after IPTG induction are shown in FIG. 13, lanes BI and AI, respectively. The gel bands marked with an arrow are the target fusion protein. The lane RF-PU in FIG. 13 shows the resulted ID 5-AAT fusion protein after refolding and purification steps.

14(c). Construction, Expression and Purification of mIL15-Linker2-mAAT (X=Asn, Z=Cys)

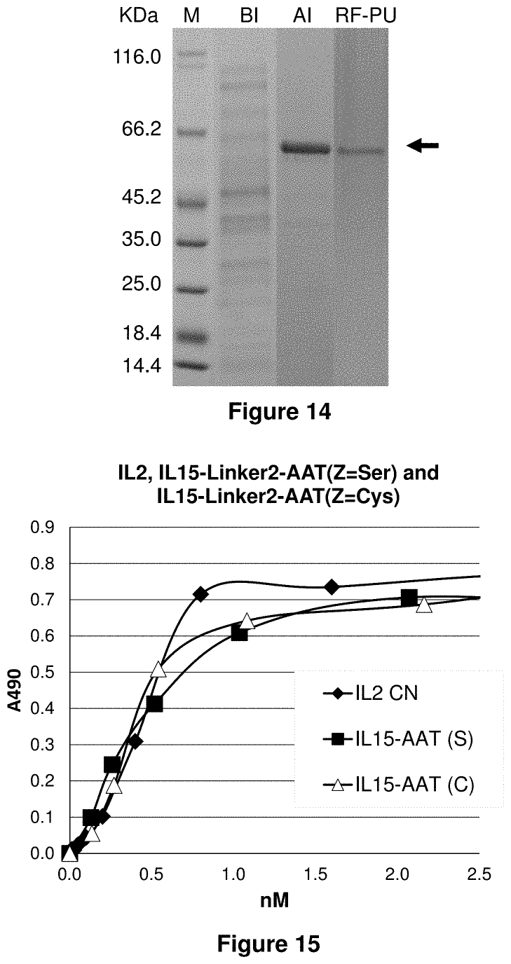

The expression vector for this fusion protein was generated by site-directed mutagenesis of the expression vector from 14(b) (SEQ ID. 27 for cDNA sequence) with the primer pair of SEQ ID. 19 and SEQ ID. 20, following the procedure described above. The resulted cDNA sequence contained Cys residue at AAT amino acid 256 (Z position). The cDNA sequence of this fusion protein mIL15-linker2-AAT(X=Asn, Z=Cys) is shown as SEQ ID. 29, which was confirmed by DNA sequencing. The sequence of the expressed protein is shown in SEQ ID.30, which was confirmed by LC-MS/MS. The expression of this construct in E. coli BL21 cells produced a very high level of the fusion protein after the IPTG induction. The expressed fusion protein was also mainly located in inclusion body. Using the procedure above, the fusion protein was refolded with a high yield similar to that from mIL15-linker2-AAT(X=Asn, Z=Ser). The refolded fusion protein was further purified with an ion-exchange column Q to a high purity. Representative data on the whole cell lysate before and after IPTG induction shown in FIG. 14, lanes BI and AI, respectively. The gel bands marked with an arrow are the target fusion protein. The lane RF-PU in FIG. 14 shows the mIL15-linker2-mAAT (X=Asn, Z=Cys) after refolding and purification steps.

15. CTLL-2 Cell Based Assay of the mIL15-Linker-mAAT Fusion Proteins

The biological activity of ID 5-AAT fusion protein can also be analyzed by CTLL-2 cell-based proliferation assay as described above. Representative data are shown in FIG. 15. Both mIL15-linker2-mAAT (X=Asn, Z=Ser) (IL15-AAT(S), solid square) and mIL15-linker2-mAAT (X=Asn, Z=Cys) (IL15-AAT(C), open triangle) fusion proteins exhibited CTLL-2 cell proliferation activities compared to recombinant IL2 reference protein. Different from the IL2-AAT fusion proteins shown in FIG. 9, activities of the ID 5-AAT fusion proteins mIL15-linker2-mAAT (X=Asn, Z=Ser) and mIL15-linker2-mAAT (X=Asn, Z=Cys) were similar based on the CTLL-2 cell-based assay.

### 16. Construction, Expression, Refolding, Purification and Characterization of the G-CSF-Linker-mAAT Fusion Proteins

### 16(a). Construction, Expression, Purification and Functional Assay of G-CSF-Linker2-Maat (Z=Ser)

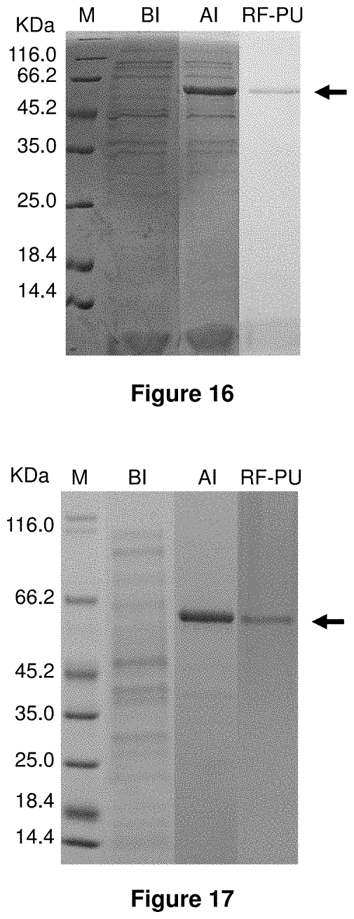

The expression vector for G-CSF-linker2-AAT(Z=Ser) fusion protein was constructed by replacing a XbaI-DraIII DNA fragment in Plasmid 2 with a synthesized DNA fragment G-CSF cDNA sequence. The resulted cDNA sequence of the fusion protein G-CSF-linker2-AAT(Z=Ser) is shown in SEQ ID. 31, which was confirmed by DNA sequencing. The sequence of the expressed fusion protein is shown in SEQ ID. 32, which is confirmed by LC-MS/MS. The expression vector produced high level of the fusion protein in E. coli BL21 cells. The expressed fusion protein was mainly located in inclusion body. Using the procedure described above, the fusion protein with Mw of about 64 kDa was refolded with a high yield, and further purified with an ion-exchange column Q to a high purity. Representative data for the fusion protein expression in E. coli BL21 whole cell lysate before, after IPTG induction and after refolding/purification were shown in FIG. 16, Lanes BI, AI and RF-PU, respectively, with the fusion protein indicated by an arrow.

### 16(b). Construction, Expression, Purification of G-CSF-Linker2-mAAT(Z=Cys)

The expression vector for the fusion protein G-CSF-linker2-mAAT (Z=Cys) was generated by site-directed mutagenesis using the expression vector described in 16(a) and a primer pair of SEQ ID. 19 and SEQ ID. 20, which resulted in a Ser to Cys mutation at AAT amino acid 256 position (Z position. The resulted cDNA sequence of the fusion protein G-CSF-linker2-AAT(Z=Cys) is shown in SEQ ID. 33, which was confirmed by DNA sequencing. The sequence of the expressed fusion protein is shown in SEQ ID. 34, which was confirmed by LC-MS/MS method described before. The fusion protein was expressed in E. coli BL21 cells at a high level. Almost all of the expressed fusion proteins were located in inclusion body as an insoluble form. The refolding procedure described above was conducted. Representative data of the G-CSF-linker2-AAT(Z=Cys) fusion protein expression, refolding and purification were shown in FIG. 17 with the total cellular proteins before IPTG induction (BI), the total cellular proteins after IPTG induction (AI) and the fusion protein after refolding and purification (RF-PU). The fusion protein band is indicated by an arrow.

### 17. M-NFS-60 Cell Based Assay of the G-CSF-Linker-mAAT Fusion Proteins

The biological activity of G-CSF-AAT fusion proteins produced above were analyzed with an M-NFS-60 cell-based proliferation assay to test cell growth stimulation in the presence of G-CSF or G-CSF-AAT fusion protein. Assay protocol is briefly described below.

G-CSF standards and G-CSF-AAT fusion proteins were each run in triplicate, in a ten-point dilution series, using a single 96-well assay plate. Starting concentration and dilution scheme were optimized to achieve a full dose-response curve with proper upper and lower asymptotes and sufficient points on the slope. Standard recombinant G-CSF control was diluted in a 2:1 ten-point dilution series with complete RPMI1640 medium. Fifty microliters of a sample were added to each well. G-CSF standard curve starting concentration was selected at 20 ng/ml.

M-NFS-60 cells were spun down and washed with RPMI1640 medium.

The cells were then resuspended in complete medium at a cell density of 6×10⁵ cells/ml. Fifty microliters (μ) of cells were added into each well in the 96-well-plates. The cells are incubated at 37° C. in a 5% CO₂ for 2 days.

After 2 days incubation, 20 μl of Cell Titer 96 Aqueous reagents (1 vol of tetrazolium compound (MTS) and 1 vol of an electron coupling reagent, phenazine ethosulfate (PES) in Dulbecco's phosphate-buffered saline.) was added to each well. After incubating the mixture at 37° C. in a 5% CO₂ for 2 h, the absorbance at 490 nm was read using a BioRad plate reader. The solution is composed of a novel

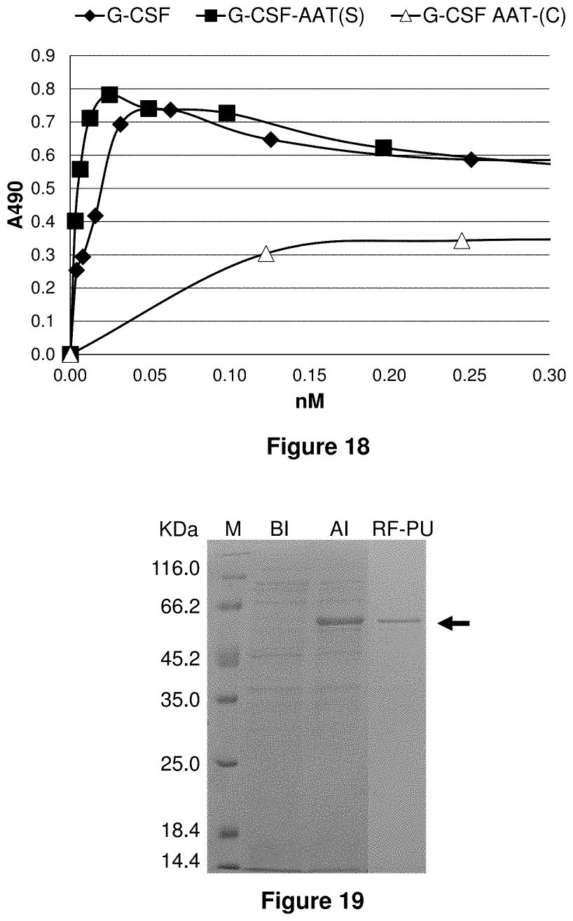

Representative data are shown in FIG. 18. Both G-CSF-linker2-mAAT (Z=Ser) (shown as G-CSF-AAT(S)) and G-CSF-linker2-mAAT (Z=Cys) (shown as G-CSF-AAT (C)) fusion proteins exhibited biological activity. The G-CSF-linker2-mAAT (Z=Ser) fusion protein had a higher activity than G-CSF-linker2-mAAT (Z=Cys) as determined by the M-NFS-60 cell proliferation assay.

### 18. Construction, Expression, Refolding, Purification and Characterization of the GM-CSF-Linker-mAAT Fusion Proteins

### 18(a). Construction, Expression and Purification of GM-CSF-Linker2-mAAT (Z=Ser)

The expression vector for GM-CSF-linker2-AAT(Z=Ser) fusion protein was constructed by replacing a XbaI-DraIII DNA fragment in Plasmid 2 with a synthesized DNA fragment GM-CSF cDNA sequence. The cDNA sequence of the fusion protein GM-CSF-linker2-AAT(Z=Ser) is shown in SEQ ID. 35, which was confirmed by DNA sequencing. The sequence of the expressed GM-CSF-linker2-AAT(Z=Ser) protein is in SEQ ID. 36, which was confirmed by LC-MS/MS using the procedure described above. The fusion protein expressed in E. coli BL21 cells at a very high level. The expressed fusion protein was found to be mainly located in inclusion body. Using the procedure described above, the fusion protein was refolded with a high yield. The refolded protein was purified with an ion-exchange column Q to homogeneity using the procedure described above. Representative data are shown in FIG. 19: lane BI was the cell lysate proteins before IPTG induction; lane AI was cell lysate proteins after IPTG induction; lane RF-PU was the refolded and purified fusion protein as indicated with an arrow.

### 18(b). Construction, Expression and Purification of GM-CSF-Linker2-mAAT (Z=Cys)

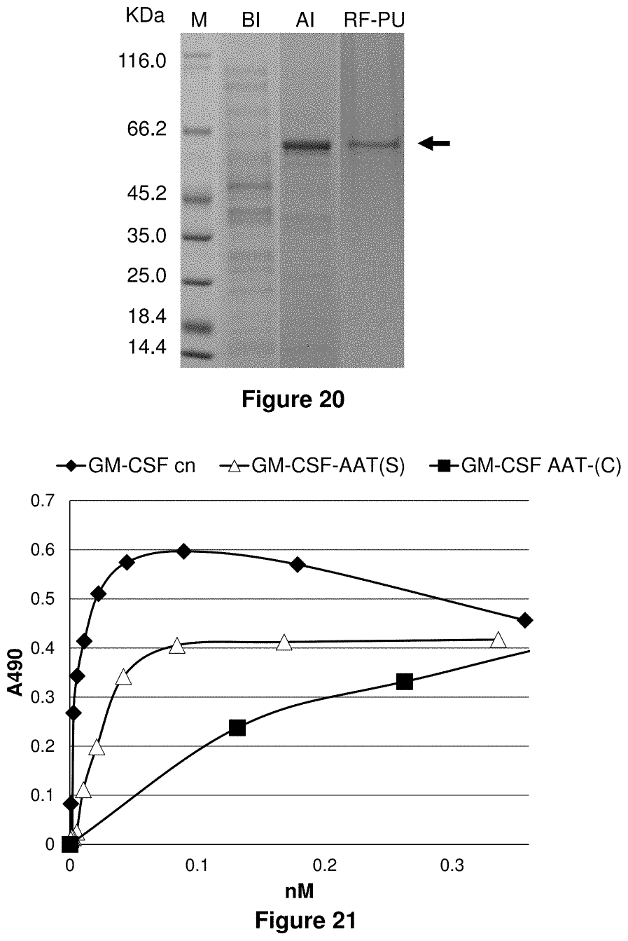

The expression vector for the fusion protein GM-CSF-linker2-mAAT (Z=Cys) was generated by site-directed mutagenesis. The sequences of the primer pair used in the mutagenesis were of SEQ ID. 19 and SEQ ID. 20, which resulted in a Ser to Cys mutation at AAT amino acid 256 position (Z position). The resulted cDNA sequence of the fusion protein GM-CSF-linker2-AAT(Z=Cys) is shown in SEQ ID. 37, which was confirmed by DNA sequencing. The sequence of the expressed fusion protein is shown in SEQ ID. 38, which was confirmed by LC-MS/MS method described above 6. The fusion protein was expressed in E. coli BL21 cells at a high level. Almost all the expressed fusion protein was located in inclusion body as an insoluble form. Refolding of the fusion protein was conducted with the procedure described above. Representative data of the GM-CSF-linker2-AAT(Z=Cys) fusion protein expression, refolding and purification were shown in FIG. 20, wherein lane BI was the total cellular proteins before IPTG induction, lane AI was the total cellular proteins after IPTG induction, and lane RF-PU was the fusion protein after refolding and purification.

### 19. Biological Activity of GM-CSF-Linker2-mAAT (Z=Ser) and GM-CSF-Linker2-Maat (Z=Cys)

Biological activities of GM-CSF-AAT fusion proteins were analyzed using the TF-1 cell-based proliferation assay, wherein the growth of TF-1 cells are dependent on granulocyte-macrophage colony-stimulating factor (GM-CSF). The procedure of the assay is described below.

TF-1 cells were from ATCC (CRL-2003). GM-CSF reference standard used in the assay as a control was the recombinant GM-CSF protein (Xiamen Tebao Bioengineering LLC). Each test and reference GM-CSF sample was run in triplicate, in a ten-point dilution series, using a single 96-well assay plate. GM-CSF standard or the fusion protein were diluted in a 2:1 ten-point dilution series with complete RPMI1640 medium. Then 50 ul of each sample were added to each well. The GM-CSF standard curve (GM-CSF cn) was with a starting concentration of 20 ng/ml.

The TF-1 cells were washed with RPMI1640 medium and then resuspended in complete medium at a cell density of 6×10⁵ cells/ml. The 50 μl cells were added into each well in 96-well-plates. The cells were incubated at 37° C. in a 5% CO₂ for 2 days. After 2 days incubation, 20 μl of Cell Titer 96 Aqueous reagents (1 vol of MTS and 1 vol of PES composed of a novel tetrazolium compound (MTS) and an electron coupling reagent, phenazine ethosulfate (PES) in Dulbecco's phosphate-buffered saline) was added to each well and incubated at 37° C. in a 5% CO₂ for 2 h. The absorbance at 490 nm was read using a BioRad plate reader.

Representative data from the cell-based assay are shown in FIG. 21. Both GM-CSF-linker2-mAAT (Z=Ser) (shown as GM-CSF-AAT(S)) and GM-CSF-linker2-mAAT (Z=Cys) (shown as GM-CSF-AAT(C)) fusion proteins exhibited stimulatory biological activities. The GM-CSF-linker2-mAAT (Z=Ser) fusion protein had a higher activity than the GM-CSF-linker2-mAAT (Z=Cys) based on the cell proliferation assay.

### 20. Construction, Expression, Refolding, Purification and Characterization of the IFNα2-Linker-mAAT Fusion Proteins

### 20(a). Construction, Expression and Purification of IFNa2-Linker2-mAAT (Z=Ser)

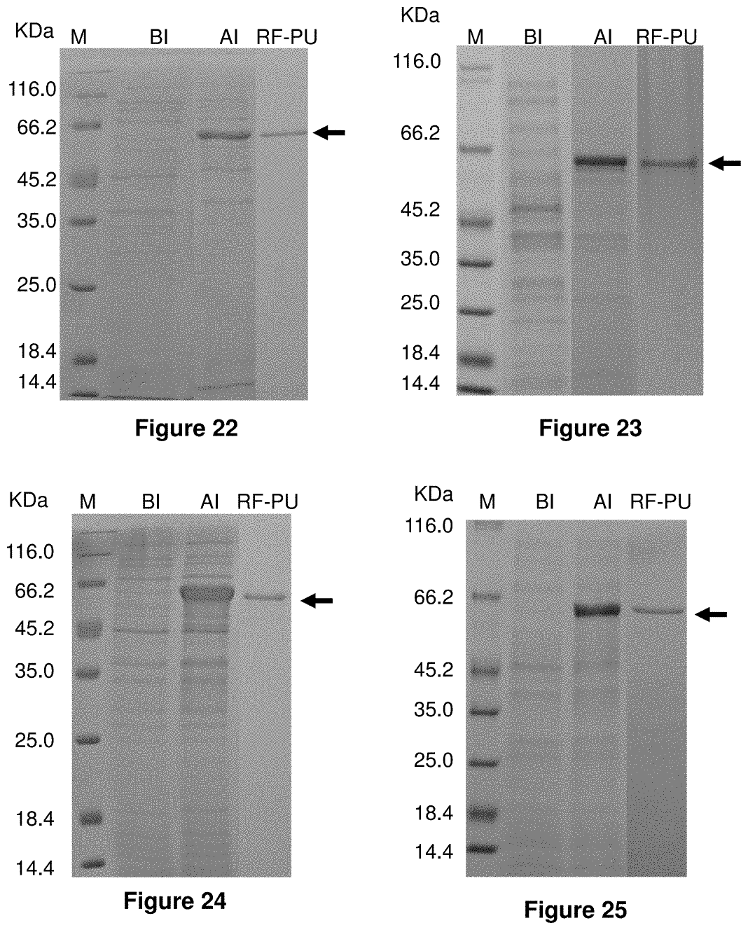

The expression vector for this IFNa2-linker2-AAT(Z=Ser) fusion was constructed by replacing a XbaI-DraIII DNA fragment in Plasmid 2 with a synthesized DNA fragment IFNα2 cDNA sequence. The synthesized cDNA sequence was optimized in codon usage for E. coli K12 expression. The cDNA sequence of the fusion protein IFNα2-linker2-AAT(Z=Ser) is listed in SEQ ID. 39, which was confirmed by DNA sequencing. The sequence of the expressed IFNα2-linker2-AAT(Z=Ser) protein is shown in SEQ ID. 40, which was confirmed by LC-MS/MS using the procedure described above. The fusion protein was expressed in E. coli BL21 cells at a very high level. The expressed fusion protein was found to be mainly located in inclusion body. Using the procedure described above, the fusion protein isolated from inclusion body was refolded with a high yield. The refolded protein was purified with an ion-exchange column Q to homogeneity as described above. Representative data for IFNα2-linker2-AAT(Z=Ser) fusion protein expression, refolding and purification are shown in FIG. 22: Lane BI was from the cell lysate protein before IPTG induction; lane AI was cell lysate proteins after IPTG induction and lane RF-PU was the refolded and purified fusion protein as indicated with an arrow.

### 20(b). Construction, Expression, Purification of IFNα2-Linker2-mAAT (Z=Cys)

The expression vector for the fusion protein IFNα2-linker2-mAAT(Z=Cys) was generated by site-directed mutagenesis from the expression vector for IFNα2-linker2-mAAT (Z=Ser) described in 20(a). The sequences of the primer pair used in the mutagenesis were SEQ ID. 19 and SEQ ID. 20. The mutagenesis changed Ser residue at AAT amino acid 256 position (Z position) to Cys residue. The resulted cDNA sequence of the fusion protein IFNα2-linker2-AAT(Z=Cys) is shown in SEQ ID. 41, which was confirmed by DNA sequencing. The sequence of the expressed fusion protein is shown in SEQ ID. 42, which was confirmed by LC-MS/MS. The fusion protein was expressed in E. coli BL21 cells at a high level. Almost all of the expressed fusion protein was located in inclusion body as an insoluble form. Refolding was conducted with the procedure described above. Representative data of the IFNα2-linker2-AAT(Z=Cys) fusion protein expression, refolding and purification were shown in FIG. 23: lane BI, the total cellular protein before IPTG induction, lane AI, the total cellular protein after IPTG induction and lane RF-PU, the fusion protein after refolding and purification.

### 21. Construction, Expression, Refolding, Purification and Characterization of the IFNβ1-Linker-mAAT Fusion Proteins

### 21(a). Construction, Expression and Purification of IFNβ1-Linker2-mAAT (Z=Ser)

The expression vector for this IFNβ1-linker2-AAT(Z=Ser) fusion was constructed by replacing a XbaI-DraIII DNA fragment in Plasmid 2 with a synthesized DNA fragment IFNβ1 cDNA sequence. The synthesized cDNA sequence was optimized in codon usage for E. coli K12 expression. The cDNA sequence of the fusion protein IFNβ1-linker2-AAT(Z=Ser) is listed in SEQ ID. 43, which was confirmed by DNA sequencing. The sequence of the expressed IFNβ1-linker2-AAT(Z=Ser) protein is shown in SEQ ID. 44, which was confirmed by LC-MS/MS. The fusion protein was expressed in E. coli BL21 cells at a very high expression level. The expressed fusion protein was found to be mainly located in inclusion body. Using the procedure described above, the fusion protein isolated from inclusion body was refolded with a high yield. The refolded protein was purified with an ion-exchange column Q to homogeneity using the procedure described above. Representative data of IFNβ1-linker2-AAT(Z=Ser) fusion protein expression, refolding and purification are shown in FIG. 24: Lane BI, the cell lysate protein before IPTG induction; lane AI, cell lysate proteins after IPTG induction and lane RF-PU, the refolded and purified fusion protein as indicated by an arrow.

### 21(b). Construction, Expression, Purification of IFNβ1-Linker2-mAAT (Z=Cys)

The expression vector for the fusion protein IFNβ1-linker2-mAAT (Z=Cys) was generated by site-directed mutagenesis from the expression vector for IFNβ1-linker2-mAAT (Z=Ser) described in 21(a). The sequences of the primer pair used in the mutagenesis were SEQ ID. 19 and SEQ ID. 20. The mutagenesis changed Ser residue at AAT amino acid 256 position (Z position) to Cys residue. The resulted cDNA sequence of the fusion protein IFNβ1-linker2-AAT(Z=Cys) is shown in SEQ ID. 45, which was confirmed by DNA sequencing. The sequence of the expressed fusion protein is shown in SEQ ID. 46, which was confirmed by LC-MS/MS. The fusion protein was expressed in E. coli BL21 cells at a high level. Almost all the expressed fusion protein was located in inclusion body as an insoluble form. Refolding was conducted with the procedure described above. Representative data of IFNβ1-linker2-AAT(Z=Cys) fusion protein expression, refolding and purification were shown in FIG. 25: lane BI, the cell lysate protein before IPTG induction; lane AI, cell lysate proteins after IPTG induction and lane RF-PU, the refolded and purified fusion protein as indicated by an arrow.

### 22. Construction, Expression, Refolding, Purification and Characterization of the GLP-1-Linker-mAAT Fusion Proteins

### 22(a). Construction, Expression, Purification and Functional Assay of GLP1-Linker2-Maat (Z=Ser)

The expression vector for this GLP1-linker2-AAT(Z=Ser) fusion was constructed by replacing a XbaI-DraIII DNA fragment in Plasmid 2 with a synthesized DNA fragment containing the GLP-1 cDNA sequence. The synthesized cDNA sequence was optimized in codon usage for E. coli K12 expression. The cDNA sequence of the fusion protein GLP1-linker2-AAT(Z=Ser) is listed in SEQ ID. 47, which was confirmed by DNA sequencing. The sequence of the expressed GLP1-linker2-AAT(Z=Ser) protein is shown in SEQ ID. 48, which was confirmed by LC-MS/MS. The fusion protein was expressed in E. coli BL21 cells at a very high expression level. The expressed fusion protein was found to be mainly located in inclusion body. Using the procedure described above, the fusion protein isolated from inclusion body was refolded with a high yield. The refolded protein was purified with an ion-exchange column Q to homogeneity using the procedure described above.

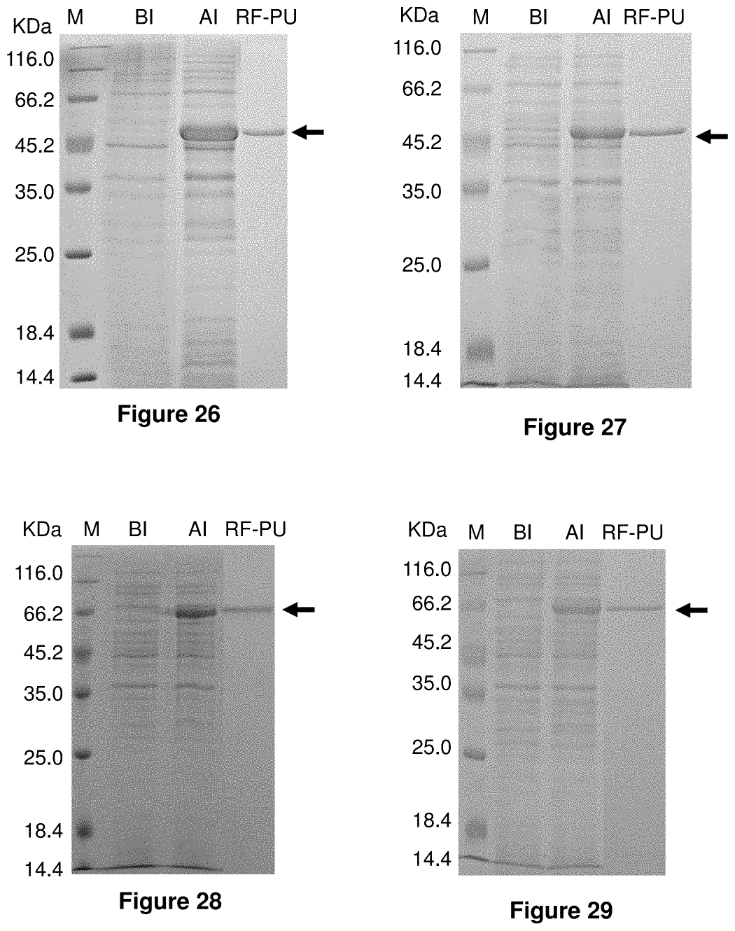

Representative data of GLP1-linker2-AAT(Z=Ser) fusion protein expression, refolding and purification are shown in FIG. 26: lane BI, the cell lysate protein before IPTG induction; lane AI, cell lysate proteins after IPTG induction and lane RF-PU, the refolded and purified fusion protein as indicated by an arrow.

### 22(b). Construction, Expression, Purification of GLP1-Linker2-mAAT (Z=Cys)

The expression vector for the fusion protein GLP1-linker2-mAAT (Z=Cys) was generated by site-directed mutagenesis from the expression vector GLP1-linker2-mAAT (Z=Ser) described in 22(a). The sequences of the primer pair used in the mutagenesis were SEQ ID. 19 and SEQ ID. 20. The mutagenesis changed Ser residue at AAT amino acid 256 position (Z position) to Cys residue. The resulted cDNA sequence of the fusion protein GLP1-linker2-AAT(Z=Cys) is shown in SEQ ID. 49, which was confirmed by DNA sequencing. The sequence of the expressed fusion protein is shown in SEQ ID. 50, which was confirmed by LC-MS/MS. The fusion protein was expressed in E. coli BL21 cells at a high level. Almost all the expressed fusion protein was located in inclusion body as an insoluble form. Refolding of the fusion protein was conducted with the procedure described above. Representative data of GLP1-linker2-AAT(Z=Cys) fusion protein expression, refolding and purification are shown in FIG. 27: lane BI, the cell lysate protein before IPTG induction; lane AI, cell lysate proteins after IPTG induction and lane RF-PU, the refolded and purified fusion protein as indicated by an arrow.

23. Construction, Expression, Refolding, Purification and Characterization of the mAAT-Linker-FGF21 Fusion Proteins
23(a). Vector Construction and Expression of mAAT-Linker2-FGF21(Z=Ser)

The cDNA for mAAT-linker2-FGF21 (FGF21 is located at the C terminal of AAT, Z=Ser) was chemically synthesized. Its cDNA sequence is shown in SEQ ID. 51. Different from other AAT fusion proteins described in this disclosure, the bioactive polypeptide FGF21 was fused to the C-terminal of AAT via a linker2 (GGGGSGGGGS) to preserve the C-terminal of the FGF21 protein that is important for the receptor binding activity. In general, the choice of N- or C-terminal fusion with the AAT can be determined based on the structure and the activity of the bioactive polypeptide.

The sequence of the synthesized cDNA for mAAT-linker2-FGF21 was confirmed by DNA sequencing. The synthesized DNA fragment also contains NaeI-BamHI restriction sites on two ends and was inserted into an E. coli expression vector PT88 digested with SspI-BamHI. The expression of the target proteins was induced by the addition of IPTG into growth media. The sequence of the expressed fusion protein is shown in SEQ ID. 52.

As shown in FIG. 28, the expression of the fusion protein before IPTG induction was very low (lane BI), and the expression level was increased after IPTG induction (lane AI). The expressed fusion protein was exclusively located in inclusion body, and the refolding was carried out using the procedure described above. The fusion protein was the purified with the procedure described above. As seen in FIG. 28, the fusion protein was purified to homogeneity as shown in the lane RF-PU. The fusion protein products were characterized by LC-MS/MS procedure described above, which confirmed the correct fusion protein sequence. Based on LC-MS/MS analysis, it was confirmed that the N-terminal Met residue was mostly retained from the fusion protein.

23(b) Vector Construction and Expression of mAAT-Linker2-FGF21(Z=Cys)

The expression vector for the fusion protein mAAT-linker2-FGF21(Z=Cys) was generated by site-directed mutagenesis from the expression vector for GLP1-linker2-mAAT (Z=Ser) described in 23(a). The sequences of the primer pair used in the mutagenesis were SEQ ID. 19 and SEQ ID. 20. The mutagenesis changed Ser residue at AAT amino acid 256 position (Z position) to Cys residue. The resulted cDNA sequence of the fusion protein mAAT-linker2-FGF21(Z=Cys) is shown in SEQ ID. 53, which was confirmed by DNA sequencing. The sequence of the expressed fusion protein is shown in SEQ ID. 54, which was confirmed by LC-MS/MS. The fusion protein was expressed in E. coli BL21 cells at a high level. Almost all the expressed fusion protein was located in inclusion body as an insoluble form. Refolding was conducted with the procedure described above. Representative data of mAAT-linker2-FGF21(Z=Cys) fusion protein expression, refolding and purification are shown in FIG. 29: lane BI, the cell lysate protein before IPTG induction; lane AI, cell lysate proteins after IPTG induction and lane RF-PU, the refolded and purified fusion protein as indicated by an arrow.

24. Construction, Expression, Refolding, Purification and Characterization of the sdAb-Linker-mAAT Fusion Proteins
24(a). Construction, Expression and Purification of sdAb-Linker2-mAAT (Z=Ser)

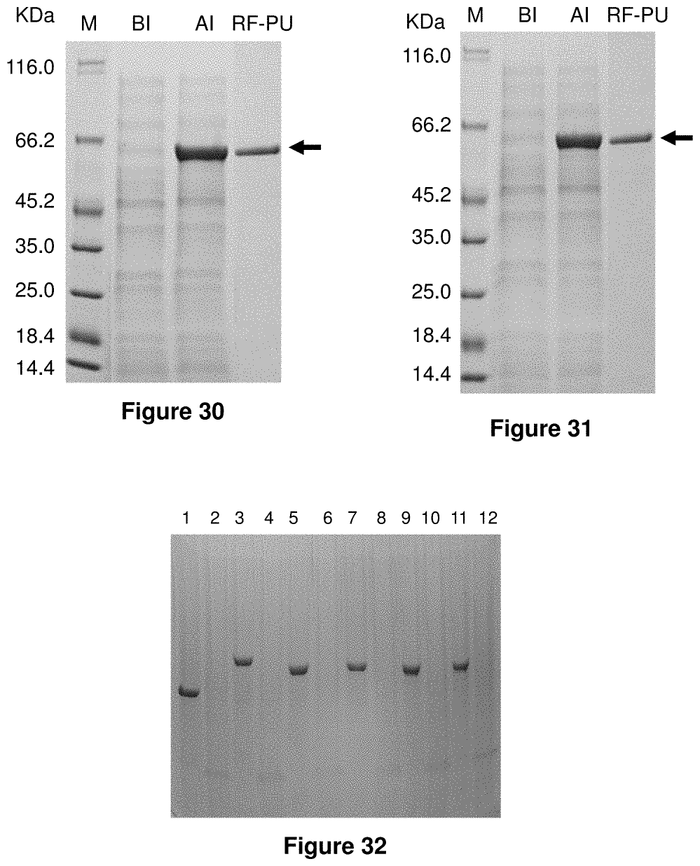

The expression vector for this sdAb-mAAT (Z=Ser) fusion protein was constructed by replacing a XbaI-DraIII DNA fragment in Plasmid 2 with a synthesized DNA fragment containing the sequence from ALX-0081, a single-domain antibody targeting von Willebrand factor. The synthesized cDNA sequence was optimized in codon usage for E. coli K12 expression. The cDNA sequence of the fusion protein sdAb-linker2-mAAT (Z=Ser) is listed in SEQ ID. 55, which was confirmed by DNA sequencing. The sequence of the expressed sdAb-linker2-mAAT (Z=Ser) fusion protein is shown in SEQ ID. 56, which was confirmed by LC-MS/MS. The fusion protein was expressed in E. coli BL21 cells at a very high expression level. The expressed fusion protein was found to be mainly located in inclusion body. Using the procedure described above, the fusion protein isolated from inclusion body was refolded with a high yield. The refolded protein was purified with an ion-exchange column Q to homogeneity using the procedure described above. Representative data on sdAb-linker2-mAAT (Z=Ser) fusion protein expression, refolding and purification are shown in FIG. 30: lane BI, the cell lysate protein before IPTG induction; lane AI, cell lysate proteins after IPTG induction and lane RF-PU, the refolded and purified fusion protein as indicated by an arrow.

24(b). Construction, Expression, Purification of sdAb-Linker2-mAAT (Z=Cys)

The expression vector for the fusion protein sdAb-linker2-mAAT (Z=Cys) was generated by site-directed mutagenesis from the expression vector for sdAb-linker2-mAAT (Z=Ser) described in 24(a). The sequences of the primer pair used in the mutagenesis were SEQ ID. 19 and SEQ ID. 20. The mutagenesis changed Ser residue at AAT amino acid 256 position (Z position) to Cys residue. The results cDNA sequence of the fusion protein sdAb-linker2-AAT(Z=Cys) is shown in SEQ ID. 57, which was confirmed by DNA sequencing. The sequence of the expressed fusion protein is shown in SEQ ID. 58, which was confirmed by LC-MS/MS. The fusion protein was expressed in E. coli BL21 cells at a high level. Almost all the expressed fusion protein was located in inclusion body as an insoluble form. Refolding was conducted with the procedure described above. Representative data of sdAb-linker2-AAT(Z=Cys) fusion protein expression, refolding and purification were shown in FIG. 31: lane BI, the cell lysate protein before IPTG induction; lane AI, cell lysate proteins after IPTG induction and lane RF-PU, the refolded and purified fusion protein as indicated by an arrow. As shown in FIG. 30 and FIG. 31, the protein expression level and refolding yield of sdAb-linker2-mAAT (Z=Ser) and sdAb-linker2-mAAT (Z=Cys) were very similar.

### 25. The Protease Inhibition Activity of the Fusion Proteins

The trypsin inhibition activities of AAT fusion proteins described above were examined using the same protease assay. As shown in FIG. 32, all the AAT fusion proteins tested were free from trypsin inhibition activities. In FIG. 32, Lane 1, 3, 5, 7, 9, 11 were purified fusion proteins GLP1-Linker2-AAT(Z=Cys), AAT(Z=Cys)-Linker2-FGF21, G-CSF-Linker2-AAT(Z=Ser), G-CSF-Linker2-AAT(Z=Cys), GM-CSF-Linker2-AAT(Z=Ser), GM-CSF-Linker2-AAT(Z=Cys), respectively, all in trypsin digestion buffer (pH=8) but with no trypsin added; Lane 2, 4, 6, 8, 10, 12 are purified protein samples GLP1-Linker-AAT1(Z=Cys), AAT1(Z=Cys)-Linker-FGF21, G-CSF-Linker-AAT1(Z=Ser), G-CSF-Linker(Z=Cys), GM-CSF-Linker-AAT(Z=Ser), GM-CSF-Linker-AAT(Z=Cys), respectively, all in trypsin digestion buffer (pH=8) but with trypsin added: trypsin=10:1 (w/w) ratio (Sequence Grade Modified Trypsin, Catalog Number V511C, Promega, Madison, Wis., USA). The fusion proteins were used as substrates for the trypsin. After 1-hour incubation at 37 C, the fusion proteins were completely digested as evidenced by the disappearance of the fusion protein bands. The results demonstrated that the fusion proteins are free from trypsin inhibition activity for both N- or C-terminal fusion, or the sequence variants at Z position.

1. A fusion protein composition comprising an AAT polypeptide or a functional variant thereof, and a bioactive polypeptide, wherein said bioactive polypeptide is covalently linked to said AAT polypeptide, covalently linked to said AAT polypeptide via a linker peptide, or a combination thereof;
wherein said AAT polypeptide comprises a mAAT polypeptide or a functional variant thereof, wherein said mAAT polypeptide or said functional variant thereof is free from cysteine amino acid residue, wherein said functional variant has at least 85% sequence identity of said mAAT polypeptide and wherein said mAAT polypeptide and said functional variant each is free from serine protease inhibitor activity.

2. The fusion protein composition of claim 1, wherein said fusion protein composition comprises said linker peptide that has an N-terminal, a C-terminal and 1-50 amino acid residues and wherein said linker peptide is positioned between said AAT polypeptide and said bioactive polypeptide.

3. The fusion protein composition of claim 2, wherein said bioactive polypeptide is linked to the N-terminal of said linker peptide and said AAT polypeptide is linked to the C-terminal of said linker peptide.

4. The fusion protein composition of claim 2, wherein said bioactive polypeptide is linked to the C-terminal of said linker peptide and said AAT polypeptide is linked to the N-terminal of said linker peptide.

5. (canceled)

6. The fusion protein composition of claim 1, wherein said fusion protein composition comprises said mAAT having a serine or an alanine mutation at a Z position in said mAAT.

7. The fusion protein composition of claim 1, wherein said bioactive polypeptide has a molecular weight in a range of from 100 to 25,000 Daltons.

8. The fusion protein composition of claim 1, wherein said bioactive polypeptide has a molecular weight in a range of from 100 to 24,000 Daltons, 0 to 3 disulfide bonds or a combination thereof.

9. The fusion protein composition of claim 1, wherein said bioactive polypeptide comprises a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof.

10. The fusion protein composition of claim 9, wherein said bioactive polypeptide comprises Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

11. The fusion protein composition of claim 10, wherein said bioactive polypeptide comprises said interleukin-2 (IL-2) or said modified IL-2 (mIL-2).

12. The fusion protein composition of claim 10, wherein said mIL-2 comprises a serine or an alanine mutation at an X position in said mIL-2.

13-20. (canceled)

21. The fusion protein composition of claim 9 further comprising a targeting agent covalently linked to said AAT or mAAT polypeptide, said bioactive polypeptide, or a combination thereof.

22. A pharmaceutical composition comprising a fusion protein and, optionally, one or more pharmaceutically acceptable carriers, said fusion protein comprising:
an AAT polypeptide or a functional variant thereof;
a bioactive polypeptide;
wherein, said bioactive polypeptide is covalently linked to said AAT polypeptide, covalently linked to said AAT polypeptide via a linker peptide, or a combination thereof; and
wherein said AAT polypeptide comprises a mAAT polypeptide or a functional variant thereof, wherein said mAAT polypeptide or said functional variant thereof is free from cysteine amino acid residue, wherein said functional variant has at least 85% sequence identity of said mAAT polypeptide and wherein said mAAT polypeptide and said functional variant each is free from serine protease inhibitor activity.

23. The pharmaceutical composition of claim 22, wherein said fusion protein comprises said linker peptide that has an N-terminal, a C-terminal and 1-50 amino acid residues and wherein said linker peptide is positioned between said AAT polypeptide and said bioactive polypeptide.

24. The pharmaceutical composition of claim 23, wherein said bioactive polypeptide is linked to the N-terminal of said linker peptide and said AAT polypeptide is linked to the C-terminal of said linker peptide.

25. The pharmaceutical composition of claim 23, wherein said bioactive polypeptide is linked to the C-terminal of said linker peptide and said AAT polypeptide is linked to the N-terminal of said linker peptide.

26. (canceled)

27. The pharmaceutical composition of claim 22, wherein said fusion protein comprises said mAAT having a serine or an alanine mutation at a Z position in said mAAT.

28. The pharmaceutical composition of claim 22, wherein said bioactive polypeptide has a molecular weight in a range of from 100 to 25,000 Daltons.

29. The pharmaceutical composition of claim 22, wherein said bioactive polypeptide has a molecular weight in a range of from 100 to 24,000 Daltons, 0 to 3 disulfide bonds or a combination thereof.

30. The pharmaceutical composition of claim 22, wherein said bioactive polypeptide comprises a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof.

31. The pharmaceutical composition of claim 30, wherein said bioactive polypeptide comprises Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

32. The pharmaceutical composition of claim 31, wherein said bioactive polypeptide comprises said interleukin-2 (IL-2) or said modified IL-2 (mIL-2).

33. The pharmaceutical composition of claim 31, wherein said mIL-2 comprises a serine or an alanine mutation at an X position in said mIL-2.

34-41. (canceled)

42. The pharmaceutical composition of claim 22, wherein said fusion protein further comprises a targeting agent covalently linked to said AAT or mAAT polypeptide, said bioactive polypeptide, or a combination thereof.

43-84. (canceled)

85. A method for treating a disease in a subject in need thereof, said method comprising administering the pharmaceutical composition of claim 22 to said subject.

86. The method of claim 85, wherein said pharmaceutical composition is administered to said subject via intravenous (IV) injection, subcutaneous (SC) injection, intramuscular (IM) injection, intradermal (ID) injection, or a combination thereof.

87. The method of claim 85, wherein said pharmaceutical composition is administered to said subject via a local injection to deliver the pharmaceutical composition into or adjacent to a disease location.

88. The method of claim 85, wherein said disease is a cancer, an autoimmune disease, diabetes, vasculitis, heart disease, virus infection, or a combination thereof.

89. The method of claim 85, wherein said pharmaceutical composition comprises said fusion protein that comprises said mAAT having a serine or an alanine mutation at a Z position in said mAAT.

90. The method of claim 85, wherein said pharmaceutical composition comprises said fusion protein that comprises said bioactive polypeptide comprises a cytokine, a modified cytokine, a peptide hormone, a modified peptide hormone, an interferon, a modified interferon, a growth factor, a modified growth factor, an antibody, a fragment of antibody, a peptide, an antigen, a neoantigen, an inhibitor, an activator, an enzyme, a binding protein, a protein, a fragment of a protein, or a combination thereof.

91. The method of claim 85, wherein said bioactive polypeptide comprises Interleukin-2 (IL-2), modified Interleukin-2 (mIL-2), Interleukin-15 (IL-15), modified Interleukin-15 (mIL-15), Granulocyte-colony stimulating factor (G-CSF), modified Granulocyte-colony stimulating factor (mG-CSF), Granulocyte-macrophage colony-stimulating factor (GM-CSF), modified Granulocyte-macrophage colony-stimulating factor (mGM-CSF), interferon alpha-2 (IFN-α2), modified interferon alpha-2 (mIFN-α2), Interferon beta-1 (IFN-β1), modified Interferon beta-1 (mIFN-β1), Glucagon-like peptide-1 (GLP-1), modified Glucagon-like peptide-1 (mGLP-1), Fibroblast growth factor 21 (FGF21), modified Fibroblast growth factor 21 (mFGF21), single domain antibody (sdAb), modified single domain antibody (msdAb), a fragment thereof, or a combination thereof.

92. The method of claim 85, wherein said bioactive polypeptide comprises said interleukin-2 (IL-2) or said modified Interleukin-2 (mIL-2).

93. The method of claim 85, wherein said mIL-2 comprises a serine or an alanine mutation at an X position in said mIL-2.
